(19) United States
(12) Patent Application Publication
(10) Pub. No.: US 2026/0108561 A1
(43) Pub. Date: Apr. 23, 2026

(54) COMPOSITION FOR CELL TRANSPLANTATION THERAPY AND THE USE THEREOF

(71) Applicant: NATIONAL UNIVERSITY OF SINGAPORE, Singapore (SG)

(72) Inventors: Suchun ZHANG, Singapore (SG); Zhifu WANG, Singapore (SG)

(73) Assignee: NATIONAL UNIVERSITY OF SINGAPORE, Singapore (SG)

(21) Appl. No.: 19/117,699

(22) PCT Filed: Oct. 3, 2023
(86) PCT No.: PCT/SG2023/050666

§ 371 Date: Apr. 2, 2025

(30) Foreign Application Priority Data
Oct. 4, 2022 (SG) 10202251263E

(51) Int. Cl. A61K 35/30; A61K 33/06; A61K 38/48; A61P 25/28

(52) U.S. Cl. CPC A61K 35/30; A61K 33/06; A61K 38/4833; A61P 25/28

(57) ABSTRACT

Disclosed is a composition for supporting survival and differentiation of neural precursor cells (NPCs) grafted into a neurological injury or disease site, the composition comprising: (a) a gel forming molecule; and (b) a chemokine receptor type 5 (CCR5) antagonist. Also disclosed is a method of treating a neurological injury or disease of a subject, comprising (a) mixing NPCs with the composition as disclosed herein; and (b) administering a mixture of the NPCs and the composition into a neurological injury or disease site of the subject, to thereby support survival and differentiation of the NPCs. Also disclosed is use of a mixture of NPCs and the composition as disclosed herein in the manufacture of a medicament for treating a neurological injury or disease of a subject, wherein the mixture is to be administered into a neurological injury or disease site of the subject, to thereby support survival and differentiation of the NPCs. Further disclosed is a kit for use in supporting survival and differentiation of NPCs grafted into a neurological injury or disease site, the kit comprising: (a) the composition as disclosed herein; (b) artificial cerebral spinal fluid (a-CSF); (c) CaCl₂; and (d) thrombin.

### FIELD OF THE INVENTION

The present invention generally relates to a composition for supporting cell survival and differentiation in cell transplantation therapy and its use thereof. In particular, the present invention relates to a composition for supporting neural precursor cells (NPCs) survival and differentiation in NPCs transplantation therapy for neurological injuries or diseases, and its use thereof.

### BACKGROUND

Neurological injuries or diseases, including stroke, traumatic brain injury, and spinal cord injury, are major causes of disability without effective treatment. For example, ischemic stroke results from an acute reduction in cerebral blood flow and has afflicted approximately 25% people over their lifetime, accounting for almost 5% of all disability-adjusted life-years and 10% of all deaths worldwide. Conventional treatment is reperfusion during the acute stage of the ischemic event. However, there is no effective treatment beyond the acute stage. There is, therefore, an unmet need to develop a treatment for neurological injuries or diseases such as ischemic stroke.

Stem cell-based approaches hold promise as cell therapies may protect the injured neurons from further damage, and/or replace the lost neurons. Transplantation of non-neural cells, such as mesenchymal stem cells (MSCs), has been initiated in clinical trials. MSCs are shown to protect neurons in the penumbral area, a brain region bordering the ischemic site and the healthy brain, by modulation of inflammation or angiogenesis. However, MSCs transplantation do not result in neuronal replacement. Since the human brain has a very limited capacity to regenerate and MSCs usually do not produce neurons, it is desirable to have neural cells as a source for cell therapy.

Indeed, transplantation with neural precursor cells (NPCs) in animal models of neurological conditions such as stroke, spinal cord injury (SCI), and Parkinson's disease (PD) has demonstrated that they can mature to become functional neurons and potentially integrate into the host brain circuitry. In animals with neurological injuries or diseases such as ischemic stroke, the neurological injury or disease site, such as the ischemic/infarct site, forms a lesion cyst or cavity that is walled off by glial scars and filled with inflammatory cells and secretions. Such an environment is hostile to transplanted NPCs, leading to poor survival of NPCs transplanted into the lesion cyst or cavity and the inability of nerve growth through the glial scar. The inhibitory milieu also promotes the grafted NPCs to differentiate into glial cells instead of neurons. Efforts are made to improve the survival of NPCs that are transplanted into the ischemic core, including overexpressing Small Ubiquitin-like Modifier (SUMO), hypoxic treatment, co-transplantation with non-neuronal cells, and hydrogels cross-linked with growth factors (such as bone morphogenetic proteins (BMP4), brain-derived neurotrophic factor (BDNF), and laminin derived motif (IKVAV)) based biomaterials. In particular, hydrogels possess anti-inflammatory action and can be resorbed by the tissue. Studies demonstrated that hyaluronan-methylcellulose exhibited anti-inflammatory properties by reducing IL-la levels in central nervous system after stroke and spinal cord injury. The hydrogels might also be modified to modulate immune response and promote angiogenesis, potentially promoting the survival and differentiation of transplanted NPCs. When encapsulated in hydrogels, NPCs transplanted into the stroke cavity could survive with limited proliferation for 2 weeks. An anti-inflammatory polarising effect of hydrogel on infiltrating microglia could indicate potential for inflammatory reprogramming of the stroke lesion, which could contribute to the neural regeneration after NPC transplantation. However, these methods fail to fill and reconstitute the damaged brain due to limited number of surviving cells. The poor cell survival even in the presence of neurotrophic support relative to that in other neurological models such as the 6-OHDA induced Parkinson's model, led to the hypothesis underlying the present invention that modification of the hostile ischemic environment, especially the inflammatory milieu, is critical for promoting the NPC survival.

Currently, most of the studies transplant NPCs into the penumbra, in order to avoid the hostile environment in the lesion cyst or cavity. Nevertheless, it creates an additional injury to the healthy brain region. It also leaves the lesion cyst or cavity unfilled, especially when the ischemic cyst is relatively large, leaving the separated brain regions unconnected. An alternative conventional method is to use a cocktail of growth factors that support the survival of NPCs transplanted into the injured spinal cord cyst. However, growth factors at thousand folds of physiological concentrations need to be used. This indeed results in a large graft that fills the lesion cyst or cavity. It however often creates an occupying tissue in the spinal cord. Furthermore, it prevents the neural progenitors from differentiating to mature neurons and glia, which is required for the grafted cells to integrate into the host tissue to achieve therapeutic goal.

NPCs transplantation into the lesion cyst or cavity would potentially fill the gap, replace the lost neural cells, and reconnect the disrupted circuitry. The efficient differentiation, or maturation, of the grafted NPCs is also critical for cell transplantation therapy to work. Thus, there is a need for a composition and a method to enable and improve the survival of the transplanted NPCs, and promote the differentiation of the transplanted NPCs into mature neurons to thereby reconstitute the neurological injury or disease site.

### SUMMARY

The present disclosure describes a composition comprising two components, a gel forming molecule and a chemokine receptor type 5 (CCR5) antagonist. In one example, the gel forming molecule and the CCR5 antagonist are FDA approved drugs, fibrinogen and Maraviroc, respectively. In the presence of the composition, the NPCs grafted into a neurological injury or disease site such as an ischemic core survive and subsequently differentiate to neurons, which reconstitutes the collapsed cortex.

In one aspect, the present disclosure refers to a composition for supporting survival and differentiation of neural precursor cells (NPCs) grafted into a neurological injury or disease site, the composition comprising:
(a) a gel forming molecule; and
(b) a chemokine receptor type 5 (CCR5) antagonist.

In another aspect, the present disclosure refers to a method of treating a neurological injury or disease of a subject, comprising
(a) mixing NPCs with the composition as disclosed herein; and
(b) administering a mixture of the NPCs and the composition into a neurological injury or disease site of the subject, to thereby support survival and differentiation of the NPCs.

In another aspect, the present disclosure refers to use of a mixture of NPCs and the composition as disclosed herein in the manufacture of a medicament for treating a neurological injury or disease of a subject, wherein the mixture is to be administered into a neurological injury or disease site of the subject, to thereby support survival and differentiation of the NPCs.

In another aspect, the present disclosure refers to a kit for use in supporting survival and differentiation of NPCs grafted into a neurological injury or disease site, the kit comprising:
(a) the composition as disclosed herein;
(b) artificial cerebral spinal fluid (a-CSF);
(c) CaCl₂); and
(d) thrombin.

Advantageously, the gel forming molecule in the composition forms a gel at 37° C. following NPCs transplantation, thus it serves as a scaffold to stabilize the NPCs grafted in the lesion cyst or cavity. The gel also prevents the CCR5 antagonist from being diluted quickly. CCR5 antagonist blocks the signalling from the inflammatory cytokines in the lesion cyst or cavity to CCR5 that is expressed on the NPCs, reducing the apoptosis of the transplanted NPCs in the inflammatory lesion cyst or cavity, and promoting the differentiation of the surviving NPCs. In addition, the composition contain FDA-approved drugs at physiological concentrations, making it applicable in clinical settings.

### BRIEF DESCRIPTION OF THE DRAWINGS

The invention will be better understood with reference to the detailed description when considered in conjunction with the non-limiting examples and the accompanying drawings, in which:

FIG. 1 illustrates the survival of grafted NPCs in the ischemic core using the composition as disclosed herein.

FIG. 1a is a schematic drawing showing the process of NPCs transplantation with fibrinogen and Maraviroc.

FIG. 1b is a schematic drawing showing the experimental procedures and timelines for the induction of ischemic stroke, cell transplantation, and tissue harvest for analysis.

FIG. 1c is a series of fluorescence microscopy images taken using Nikon Ti2 Confocal microscope, showing the immunostaining for Glial Fibrillary Acidic Protein (GFAP) in stroke mice transplanted with NPCs in the presence of artificial cerebral spinal fluid (a-CSF) alone, Maraviroc alone, fibrinogen alone, or the composition at day 7 post transplantation, showing the GFP⁺ grafted cells (green) in the lesion cavity (marked by dash outline) and GFAP⁺ glial scar surrounding the lesion cavity. Dotted lines outline the ischemic core. Scale bar, 200 μm.

FIG. 1d is a series of fluorescence microscopy images of cortical slices taken using Nikon Ti2 Confocal microscope, showing the immunoreactivity of cleaved-caspase3 (red) in grafted cells at 7 days after transplantation. Separate fluorescent channels are shown below the microcopy images. Scale bar, 100 μm.

FIG. 1e is a bar graph showing the quantification of the proportion of cleaved-caspase3⁺ cells in GFP⁺ cells. n=4 mice per group. Data are mean±SEM.

FIG. 1f is a series of fluorescence microscopy images taken using Nikon Ti2 Confocal microscope, illustrating the immunostaining for DCX and SOX2 in stroke mice transplanted with NPCs at 7 days after transplantation. Yellow arrowhead indicates surviving grafts. Dotted lines outline the ischemic core. Scale bar, 200 km.

FIG. 1g is a dot plot which illustrates the number of DCX⁺ or SOX2⁺ cells in GFP⁺ cells in different groups. n=5 mice for the cocktail/composition group, n=4 mice for all other three groups. Data are mean±SEM.

FIG. 2 illustrates the maturation of the grafted NPCs in the ischemic core using the composition as disclosed herein.

FIG. 2a is a schematic drawing showing the procedures and timelines for the induction of ischemic stroke, cell transplantation, and tissue harvest for analysis.

FIG. 2b is a fluorescence microscopy image taken using Nikon Ti2 Confocal microscope, providing an overview of transplanted human cells (labelled by STEM121) in the ischemic core (surrounded by GFAP⁺ glial scar) of the cortex in stroke mice transplanted with the cocktail/composition at 30 days post transplantation (30-dpt). Separate channels are shown on the right. Scale bar, 1 mm. LV, lateral ventricle. cc, corpus callosum.

FIG. 2c is a whole-mount view of brains from mice with (bottom panel) or without (top panel) transplantation of NPCs. Dotted lines and black arrowheads indicate the injured or transplant site. Scale bar, 2 mm.

FIG. 2d shows serial coronal slices which demonstrate that STEM121⁺ cells fill up the stroke cavity at 30 days post transplantation (30-dpt). Scale bar, 1 mm.

FIGS. 2e and 2f are fluorescence microscopy images taken using Nikon Ti2 Confocal microscope, showing the immunostaining for neurofilament (NF), which show the expression of NF in grafted cells (GFP⁺ cells) at 30-dpt. Magnified images in FIG. 2f show that GFP cells are NF positive. Scale bar, 200 μm in FIG. 2e, 100 μm in FIG. 2f.

FIG. 2g is a pie chart showing the quantification of the percentage of NF⁺ cells in GFP⁺ cells. n=4 mice. Data are mean±SEM.

FIG. 2h are fluorescence microscopy images taken using Nikon Ti2 Confocal microscope, showing the immunostaining for STEM121 and NeuN showing that grafted cells differentiate to mature neurons at 30 days post transplantation (30-dpt). Scale bar, 1 mm. LV, lateral ventricle. cc, corpus callosum.

FIG. 2i is a bar graph showing the number of GFP⁺ NeuN⁺ or GFP⁺ NeuN⁻ cells. n=5 mice. Data are mean±SEM.

FIG. 2j-2l are images of areas indicated in FIG. 2h showing grafted cells at the border (FIG. 2j), upper layer (FIG. 2k) and deep layer (FIG. 2l). Scale bar, 100 μm.

FIG. 2m is a bar graph showing the quantification of the percentage of NeuN⁺ cells in grafted cells in the upper layer and deep layer. n=4 mice. Data are mean±SEM. p=0.0013. **p<0.01.

FIG. 3 illustrates the glial reaction and vascularization in the transplanted brain.

FIG. 3a is a series of fluorescence microscopy images taken using Nikon Ti2 Confocal microscope, showing the immunostaining for Iba1 and GFAP in the mice brain transplanted with NPCs in sham, a-CSF alone, Maraviroc alone, fibrinogen alone, or the composition at 30 days after transplantation. Scale bar, 200 μm. cc, corpus callosum.

FIG. 3b is a magnified view showing the differential immunoreactivity for Iba1 and GFAP in mice with different treatments. Asterisks indicate ischemic core. Scale bar, 200 μm.

FIG. 3c is a series of fluorescence microscopy images taken using Nikon Ti2 Confocal microscope, showing the immunostaining for CSPG and S100β, which shows glial reaction in mice with composition treatment as compared with other groups. Asterisks indicate ischemic core. Scale bar, 200 μm.

FIG. 3d-3f are bar graphs showing the quantification of the indicated fluorescence intensity (normalized to the intact region) in the lesion site 30 days after transplantation. n=5 mice per group. Data are mean±SEM. **p=0.0017 in FIG. 3d, *p=0.0372 in FIG. 3e, ***p=0.0004 in FIG. 3f.

FIG. 3g is a bar graph showing the quantification of parenchymal volume in the injured area surrounded by GFAP⁺ cells. n=5 mice per group. Data are mean±SEM. ***p=0.0009.

FIG. 3h is a series of fluorescence microscopy images taken using Nikon Ti2 Confocal microscope, showing the immunostaining for STEM121 and laminin showing vascularization in the grafts at 30 days after transplantation. Scale bar, 200 μm.

FIG. 3i is a bar graph showing the quantification of the vascular density in the grafts and intact region. n=5 mice. Data are mean±SEM. ns p=0.1146.

FIG. 4 illustrates the change of chemokine ligands (CCL) and chemokine receptor type 5 (CCR5) expression in the transplanted brain.

FIG. 4a is a schematic drawing showing the experimental strategy for FIG. 4b-4f.

FIG. 4b are western blot images showing CCR5, CCL3, CCL4, and CCL5 protein expression in peri- and infarct cortex at 2, 14, and 44 days post stroke (dps).

FIG. 4c-4f are bar graphs showing the quantification of CCL3 (FIG. 4c), CCL4 (FIG. 4d), CCL5 (FIG. 4e), and CCR5 (FIG. 4f) protein expression normalized to GAPDH. n=3 mice per group. Data are mean±SEM. *p=0.0136 in FIG. 4c, *p=0.0241 in FIG. 4d, *p=0.0499 in FIG. 4e, *p=0.0244 in FIG. 4f.

FIG. 4g is a schematic drawing showing the experimental strategy for FIG. 4h-4l.

FIG. 4h are western blot images showing CCR5, CCL3, CCL4, and CCL5 protein expression in graft and infarct area at 44 days post stroke.

FIG. 4i-4l are bar graphs showing quantification of CCL3 (FIG. 4i), CCL4 (FIG. 4j), CCL5 (FIG. 4k), and CCR5 (FIG. 4l) protein expression normalized to GAPDH. n=3 mice per group in FIG. 4i-4k, n=4 mice per group in FIG. 4l. Data are mean±SEM. *p=0.0174 in FIG. 4i, **p=0.0046 in FIG. 4j, **p=0.0079 in FIG. 4k, **p=0.0066 in FIG. 4l.

FIG. 5 illustrates the expression and regulation of CCR5 in NPCs.

FIG. 5a-5c are fluorescence microscopy images taken using Nikon Ti2 Confocal microscope, showing the immunostaining for CCR5 with SOX2 (FIG. 5a), DCX (FIG. 5b), and NeuN (FIG. 5c) showing the expression of CCR5 in NPCs, immature and mature neurons. Scale bar, 200 μm.

FIG. 5d is a bar graph with western blot images showing the expression levels of CCR5 on NPCs, immature neurons, and mature neurons. n=3 samples per group. Data are mean±SEM and relative to GAPDH. **p=0.0094.

FIG. 5e is a schematic drawing showing the experimental strategy for FIG. 5f-k.

FIG. 5f are western blot images showing CCR5 expression levels on NPCs with or without CCR5-shRNA in the presence or absence CCLs.

FIG. 5g-5h are fluorescence microscopy images taken using Nikon Ti2 Confocal microscope. They are the immunostaining for SOX2 and CCR5 showing the expression of CCR5 on NPCs with vehicle (FIG. 5g) or CCR5-shRNA (FIG. 5h). Scale bar, 200 μm.

FIG. 5i are western blot images showing the expression levels of CCR5 on the indicated cells.

FIG. 5j are fluorescence microscopy images of NPCs taken using Nikon Ti2 Confocal microscope, showing the number of apoptotic NPCs (TUNEL⁺) induced by CCLs in the absence or presence of CCR5-shRNA. Scale bar, 200 μm.

FIG. 5k is a dot plot showing the quantification of the percentage of TUNEL⁺ cells in NPCs. n=5 samples per group. Data are mean±SEM. ****p<0.0001.

FIG. 5l is a schematic drawing showing that blocking the CCR5 activation feedback mitigates the apoptosis of NPCs.

FIG. 6 illustrates the establishment of ischemic stroke by photothrombosis and preparation of NPCs for transplantation.

FIG. 6a is a line graph showing in vitro maraviroc release profile of the cocktail gel and free drug.

FIG. 6b is an image showing triphenyl tetrazolium chloride (TTC) staining on brain slices showing the infarct area at 3 days post stroke (dps). Black arrowheads indicate the infarct area. Scale bar, 1 mm.

FIG. 6c-6e are fluorescence microscopy images taken using Nikon Ti2 Confocal microscope, of the immunostaining for cortical markers Brn2 (upper layer, c), Ctip2 (deep layer, d), and Foxp2 (deep layer, e) showing the different subtypes of cortical progenitors for transplantation. Scale bar, 200 μm.

FIG. 6f is a bar graph showing the quantification of the percentage of indicated NPCs in GFP⁺ cells. n=3 samples. Data are mean±SEM.

FIG. 6g are images of dissociated GFP⁺ NPCs before transplantation. Scale bar, 200 μm.

FIG. 6h is a fluorescence microscopy image taken using Nikon Ti2 Confocal microscope, of the immunostaining for astrocyte markers GFAP and S100β showing the ischemic core is surrounded by reactive astrocytes at 14-dps. Dotted lines indicate the ischemic core and corpus callosum (cc). Scale bar, 200 μm.

FIG. 6i is a fluorescence microscopy image taken using Nikon Ti2 Confocal microscope, of the immunostaining for neurite markers NF and Microtubule-associated protein 2 (MAP2) on the cortical sections showing the injured cortex has been collapsed without neurites at 30-dps. Scale bar, 200 μm.

FIG. 7 illustrates the survival and proliferation of the grafted NPCs.

FIG. 7a are fluorescence microscopy images taken using Nikon Ti2 Confocal microscope, of the immunostaining for NeuN showing that the NPCs (GFP⁺) were transplanted in the ischemic core (NeuN⁻) at 7 days after transplantation. Scale bar, 200 μm. PI, peri-infarct. cc, corpus callosum.

FIG. 7b are fluorescence microscopy images taken using Nikon Ti2 Confocal microscope, of the immunostaining for Ki67 showing the expression of Ki67 in grafted cells at 7 days after transplantation. The boxed areas are magnified in the bottom panel. Yellow arrowheads indicate Ki67 and GFP co-labelled cells. Scale bar, 200 μm.

FIG. 7c is a bar graph showing the quantification of the percentage of Ki67⁺ cells in grafted cells (GFP⁺). n=4 mice per group. Data are mean±SEM.

FIG. 8 illustrates the survival of grafted cells in the ischemic core at 30 dpt.

FIG. 8a are fluorescence microscopy images taken using Nikon Ti2 Confocal microscope, showing the immunostaining for NeuN and DCX in the mice transplanted with NPCs (GFP⁺) in the indicated medium, showing no GFP⁺ cell survival. Scale bar, 200 μm.

FIG. 8b are images of the grafts in the cocktail/composition group showing the expression of Ki67 in grafted cells (GFP⁺) at 30 days after transplantation. Scale bar, 1 mm.

FIG. 8c is a pie chart showing the quantification of the percentage of Ki67⁺ cells in the grafted cells. n=4 mice.

FIG. 8d are fluorescence microscopy images taken using Nikon Ti2 Confocal microscope, showing the immunostaining for SOX9 in the mice transplanted with NPCs in the cocktail/composition group at 30 days after transplantation. Scale bar, 1 mm.

FIG. 8e is a pie chart showing the quantification of the percentage of SOX9⁺ cells in the grafted cells. n=4 mice. cc, corpus callosum.

FIG. 8f shows immunostaining for GFP and laminin showing the collapsed cortex of four groups at one month post transplantation. Scale bar, 500 μm. cc, corpus callosum.

FIG. 9 illustrates that grafted cells with the cocktail treatment project axons out of the ischemic core at 30 days after transplantation.

FIG. 9a shows immunostaining for STEM121 and GFAP in the mice transplanted with cocktail and NPCs at 30 days after transplantation, showing that axons grew out through the glial scar. Scale bar, 200 μm.

FIG. 9b shows immunostaining for STEM121 and glutamatergic marker VGluT1 showing that grafted cells differentiated to glutamatergic neurons. Scale bar, 10 μm.

FIG. 9c shows immunostaining for STEM121, synapsin (pre-synaptic marker), and psd95 (post-synaptic marker) at the undamaged region adjacent to injured site, showing that neurites of grafted neurons form synapses (white arrows) with host neurons. Scale bar, 20 μm. cc, corpus callosum.

FIG. 10 illustrates results of behaviour tests which were performed at −14 days (pre-stroke), 0 days, 14 days, and 30 days post transplantation.

FIG. 10a shows quantification of the latency to fall on the rotarod test showing the motor recovery at 30 days post transplantation. n=9 mice for maraviroc and fibrinogen groups, n=10 mice for all other groups. Data are mean±SEM. * p=0.034 (cocktail vs control).

FIG. 10b shows data on motor performances assessed with grid-walking tests. n=9 mice for all groups in b. Data are mean±SEM. n.s. p=0.053 (cocktail vs control).

FIG. 11 illustrates that CCLs induce apoptosis of NPCs.

FIG. 11a are fluorescence microscopy images taken using Nikon Ti2 Confocal microscope, of the immunostaining for SOX2, STEM121, and cleaved-caspase3 showing the differential expression of cleaved-caspase3 in the NPCs with the indicated treatment. Scale bar, 200 μm.

FIG. 11b is a bar graph showing the quantification of the percentage of cleaved-caspase3⁺ cells showing apoptosis in the presence of high (300 ng/mL) and low concentration (10 ng/mL) of CCLs. n=5 samples for low CCLs group, n=4 samples for other four groups. Data are mean±SEM. **p=0.0058.

FIG. 11c are phase contrast images of the live NPCs and immature neurons under the indicated treatment. Scale bar, 200 μm.

FIG. 12 illustrates the effects of CCR5 inhibition on the survival of NPCs.

FIG. 12a are fluorescence microscopy images taken using Nikon Ti2 Confocal microscope, showing fluorescent staining for CCR5, STEM121, and TUNEL in the NPCs with the indicated treatment. Scale bar, 200 μm.

FIG. 12b is a bar graph showing the quantification of the proportion of the apoptotic cells (TUNEL⁺) in NPCs in the presence of shRNA or maraviroc. n=3 samples per group. Data are mean±SEM. **p=0.0064, ****p<0.0001.

FIG. 13 illustrates immunostaining for hNCAM (human neuronal marker) in the ischemic mouse brain. It indicates the projection of grafted human neurons from the cortex to brain stem. The corresponding magnifications are shown in the right panel. Scale bar: 1 mm.

FIG. 14 illustrates immunostaining for hNCAM in the spinal cord of the ischemic mouse transplanted with human neurons. It indicates the axons of the transplanted neurons project to the spinal cord. The corresponding magnifications are shown in the panel A and B. Scale bar: 200 μm.

### DETAILED DESCRIPTION

The present disclosure describes a composition comprising FDA-approved drugs, such as fibrinogen and CCR5 inhibitor Maraviroc, that supports the survival and differentiation of NPCs grafted into a neurological injury or disease site in the brain, leading to filling the lesion gap in the brain, replacing the lost cells, reconnecting the disrupted neural circuits and thus behavioral recovery in a subject suffering from a neurological injury or disease. This composition may be applied for cell transplantation therapy in neurological injuries or diseases, such as stroke, traumatic brain injury, spinal cord injury, multiple sclerosis, Alzheimer's disease, Parkinson's disease, Huntington disease, Amyotrophic lateral sclerosis (ALS) and other neurological conditions characterized by inflammation.

In one aspect, the present disclosure refers to a composition for supporting survival and differentiation of neural precursor cells (NPCs) grafted into a neurological injury or disease site, the composition comprising: (a) a gel forming molecule; and (b) a chemokine receptor type 5 (CCR5) antagonist.

As used herein, the term “support” refers to maintaining, promoting, increasing or improving the proportion of NPCs that survive and differentiate to functional neurons and glial cells after being grafted into a neurological injury or disease site.

As used herein, the term “survival” refers to the viability of cells, in this case, NPCs, characterized by the capacity to perform certain functions such as metabolism, growth, reproduction, some form of responsiveness, and adaptability. In one example, NPCs survive and are viable if the NPCs grafted into a neurological injury or disease site do not express cleaved-caspase3. In another example, NPCs survive and are viable if the NPCs grafted into a neurological injury or disease site are negative for Terminal deoxynucleotidyl transferase dUTP nick end labeling (TUNEL) staining. In one example, at least 80% of NPCs are viable after being grafted into a neurological injury or disease site, in the presence of the composition as disclosed herein. In another example, at least 85% of NPCs are viable after being grafted into a neurological injury or disease site, in the presence of the composition as disclosed herein. In another example, at least 90% of NPCs are viable after being grafted into a neurological injury or disease site, in the presence of the composition as disclosed herein. In another example, at least 95% of NPCs are viable after being grafted into a neurological injury or disease site, in the presence of the composition as disclosed herein. In another example, at least 99% of NPCs are viable after being grafted into a neurological injury or disease site, in the presence of the composition as disclosed herein. In another example, about 100% of NPCs are viable after being grafted into a neurological injury or disease site, in the presence of the composition as disclosed herein.

As used herein, the term “differentiation” refers to the alteration of NPCs to a more specialized cell type, such as neurons, and/or glial cells. In one example, the NPCs after being grafted into a neurological injury or disease site differentiate to neurons, in the presence of the composition as disclosed herein. In another example, the differentiated neurons are selected from the group consisting of bipolar, multipolar, and pseudounipolar neurons. In another example, the differentiated neuron is a bipolar neuron which has one axon and one dendrite extending from the soma. In another example, the differentiated neuron is a multipolar neuron which contains one axon and multiple dendrites. Multipolar neurons can be found in the central nervous system (CNS, made up of the brain and spinal cord). In another example, a multipolar neuron is a Purkinje cell in the cerebellum, which has many branching dendrites but only one axon. In another example, the differentiated neuron is a pseudounipolar neuron which has a single process that extends from the soma, but this process later branches into two distinct structures, like a bipolar cell. In one example, a pseudounipolar neuron is a sensory neuron which has an axon that branches into two extensions: one connected to dendrites that receive sensory information and another that transmits this information to the spinal cord. In another example, the NPCs after being grafted into a neurological injury or disease site differentiate to glial cells, in the presence of the composition as disclosed herein. In another example, the differentiated glial cells are selected from the group consisting of astrocytes, microglia, oligodendrocytes, radial glia, and ependymal cells. In another example, the differentiated glial cells are astrocytes, which make contact with both capillaries and neurons in the CNS to provide nutrients and other substances to neurons, regulate the concentrations of ions and chemicals in the extracellular fluid, and provide structural support for synapses. In another example, the differentiated glial cells are microglia, which scavenge and degrade dead cells and protect the brain from invading microorganisms. In another example, the differentiated glial cells are oligodendrocytes, which form myelin sheaths around axons in the CNS. In another example, the differentiated glial cells are radial glia which serve as scaffolds for developing neurons as they migrate to their end destinations. In another example, the differentiated glial cells are ependymal cells, which line fluid-filled ventricles of the brain and the central canal of the spinal cord.

In one example, the NPCs after being grafted into a neurological injury or disease site differentiate to neurons, in the presence of the composition as disclosed herein. In one example, 85%-95% of NPCs differentiate to neurons after being grafted into a neurological injury or disease site, in the presence of the composition as disclosed herein. In another example, 85%-90% of NPCs differentiate to neurons after being grafted into a neurological injury or disease site, in the presence of the composition as disclosed herein. In another example, 90%-95% of NPCs differentiate to neurons after being grafted into a neurological injury or disease site, in the presence of the composition as disclosed herein. In another example, about 85% of NPCs differentiate to neurons after being grafted into a neurological injury or disease site, in the presence of the composition as disclosed herein. In another example, about 86% of NPCs differentiate to neurons after being grafted into a neurological injury or disease site, in the presence of the composition as disclosed herein. In another example, about 87% of NPCs differentiate to neurons after being grafted into a neurological injury or disease site, in the presence of the composition as disclosed herein. In another example, about 88% of NPCs differentiate to neurons after being grafted into a neurological injury or disease site, in the presence of the composition as disclosed herein. In another example, about 89% of NPCs differentiate to neurons after being grafted into a neurological injury or disease site, in the presence of the composition as disclosed herein. In another example, about 90% of NPCs differentiate to neurons after being grafted into a neurological injury or disease site, in the presence of the composition as disclosed herein. In another example, about 91% of NPCs differentiate to neurons after being grafted into a neurological injury or disease site, in the presence of the composition as disclosed herein. In another example, about 92% of NPCs differentiate to neurons after being grafted into a neurological injury or disease site, in the presence of the composition as disclosed herein. In another example, about 93% of NPCs differentiate to neurons after being grafted into a neurological injury or disease site, in the presence of the composition as disclosed herein. In another example, about 94% of NPCs differentiate to neurons after being grafted into a neurological injury or disease site, in the presence of the composition as disclosed herein. In another example, about 95% of NPCs differentiate to neurons after being grafted into a neurological injury or disease site, in the presence of the composition as disclosed herein. In another example, 5%-15% of NPCs differentiate to glial cells after being grafted into a neurological injury or disease site, in the presence of the composition as disclosed herein. In another example, 5%-10% of NPCs differentiate to glial cells after being grafted into a neurological injury or disease site, in the presence of the composition as disclosed herein. In another example, 10%-15% of NPCs differentiate to glial cells after being grafted into a neurological injury or disease site, in the presence of the composition as disclosed herein. In another example, about 5% of NPCs differentiate to glial cells after being grafted into a neurological injury or disease site, in the presence of the composition as disclosed herein. In another example, about 10% of NPCs differentiate to glial cells after being grafted into a neurological injury or disease site, in the presence of the composition as disclosed herein. In another example, about 15% of NPCs differentiate to glial cells after being grafted into a neurological injury or disease site, in the presence of the composition as disclosed herein.

In one example, the NPCs differentiated to neurons and/or glial cells about one month to six months after being grafted into a neurological injury or disease site, in the presence of the composition as disclosed herein. In another example, the NPCs differentiated to neurons and/or glial cells about 30 days after being grafted into a neurological injury or disease site, in the presence of the composition as disclosed herein. In another example, the NPCs differentiated to neurons and/or glial cells about 40 days after being grafted into a neurological injury or disease site, in the presence of the composition as disclosed herein. In another example, the NPCs differentiated to neurons and/or glial cells about 50 days after being grafted into a neurological injury or disease site, in the presence of the composition as disclosed herein. In another example, the NPCs differentiated to neurons and/or glial cells about 60 days after being grafted into a neurological injury or disease site, in the presence of the composition as disclosed herein. In another example, the NPCs differentiated to neurons and/or glial cells about three months after being grafted into a neurological injury or disease site, in the presence of the composition as disclosed herein. In another example, the NPCs differentiated to neurons and/or glial cells about four months after being grafted into a neurological injury or disease site, in the presence of the composition as disclosed herein. In another example, the NPCs differentiated to neurons and/or glial cells about five months after being grafted into a neurological injury or disease site, in the presence of the composition as disclosed herein. In another example, the NPCs differentiated to neurons and/or glial cells about six months after being grafted into a neurological injury or disease site, in the presence of the composition as disclosed herein. In one example, the NPCs differentiated neurons project axons to CNS tissues such as the brainstem, the spinal cord, and the contralateral cortex, reconnecting the ischemic cortex with the rest of the brain, about six months after being grafted, in the presence of the composition as disclosed herein.

The survival and differentiation of the NPCs grafted into a neurological injury or disease site is supported by the composition as disclosed herein comprising a gel forming molecule and a CCR5 antagonist. In one example, the gel forming molecule is fibrinogen. The fibrinogen molecule is a 340-kDa homodimeric glycoprotein consisting of 2Aα, 2Bβ, and 2γ polypeptide chains linked by 29 disulfide bridges. In the presence of thrombin, thrombin cleaves fibrinopeptides to form fibrin monomers. These monomers then polymerize in a half-staggered arrangement to form fibrin protofibrils and ultimately the fibrin network, an insoluble gel. Further, calcium decreases the time required for fibrin formation from fibrinogen by markedly accelerating the phase of fibrin monomer polymerization. Advantageously, once fibrinogen forms a gel in the presence of thrombin or calcium, the gel provide a scaffold to stabilize the grafted NPCs at the neurological injury or disease site. In addition, fibrinogen is neurotrophic and it supports the growth of the transplanted NPCs. Further, the fibrinogen formed gel prevents rapid dilution and/or degradation of Maraviroc, supporting the survival of transplanted NPCs.

In one example, the fibrinogen in the composition as disclosed herein has a concentration of 5 mg/mL-30 mg/mL. In another example, the fibrinogen in the composition as disclosed herein has a concentration of about 5 mg/mL. In another example, the fibrinogen in the composition as disclosed herein has a concentration of about 9 mg/mL. In another example, the fibrinogen in the composition as disclosed herein has a concentration of about 10 mg/mL. In another example, the fibrinogen in the composition as disclosed herein has a concentration of about 15 mg/mL. In another example, the fibrinogen in the composition as disclosed herein has a concentration of about 20 mg/mL. In another example, the fibrinogen in the composition as disclosed herein has a concentration of about 25 mg/mL. In another example, the fibrinogen in the composition as disclosed herein has a concentration of about 30 mg/mL.

In another example, the gel forming molecule is agarose. In another example, the gel forming molecule is collagen. In another example, the gel forming molecule is gelatin. In another example, the gel forming molecule is chitosan. In another example, the gel forming molecule is alginate. In another example, the gel forming molecule is fibrin. In another example, the gel forming molecule is hyaluronic acid. In another example, the gel forming molecule is laminin. In another example, the gel forming molecule is a degradable polymer selected from the group consisting of poly(glycolic acid) (PGA), poly(caprolactone) (PCL), poly(lactic acid) (PLA), poly(lactic acid-co-glycolic acid) (PLGA), and poly(ethylene glycol) (PEG).

The composition disclosed herein comprises a CCR5 antagonist. CCR5 is an inflammatory chemokine receptor. In the immune system, CCR5 is predominantly expressed on T cells, macrophages, dendritic cells, and eosinophils. Effector CCR5⁺ T cells are directed to sites of infection and inflammation by chemokines produced in local tissues and activated innate immune cells at the site, leading to a cascade of innate immune response. CCR5 is one of the receptors for chemokine ligands 3 (CCL3), CCL4 and CCL5. CCL3 and CCL4 are two protein components of macrophage inflammatory protein 1 (MIP), also named MIP1-alpha and beta, respectively. CCR5 is expressed on neural stem cells (NSCs) or neural progenitor cells but not mature neurons. As a chemokine receptor, in the case of a neurological injury or disease, it helps the recruitment of CCR5⁺ cells to the neurological injury or disease site with CCR5 ligands (e.g. chemokines resulting from inflammation). Hence, NSCs or neural progenitor cells tend to form clusters or neural rosettes at the neurological injury or disease site, which keeps the NSCs or neural progenitor cells in an immature stem cell or progenitor state. If the CCR5 signaling is blocked, less clustering or more even distribution would occur so that the cells will differentiate or mature.

As used herein, the term “CCR5 antagonist” refers to a molecule which inhibits or attenuates or decreases the biological activity of CCR5, or decreases the protein level of CCR5. In one example, a CCR5 antagonist is a molecule which inhibits or attenuates or decreases the biological activity of CCR5, by interfering with interaction of the CCR5 with another molecule, such as its ligand, CCL3/4/5. In one example, a CCR5 antagonist is a molecule which inhibits or attenuates or decreases the biological activity of CCR5, by acting on components of the biological pathway in which CCR5 participates. In another example, a CCR5 antagonist is a molecule which decreases the expression of the gene encoding the CCR5, thus decreases the protein level of CCR5. A CCR5 antagonist may be a molecule selected from the group consisting of a small molecule, a nucleic acid, an antibody, an anticalin, a carbohydrate, and any other compound or composition which inhibits or attenuates or decreases the activity of CCR5 either by directly interacting with CCR5 or by acting on components of the biological pathway in which CCR5 participates, or by decreasing the protein expression level of CCR5.

In one example, the CCR5 antagonist is a small molecule. In another example, the CCR5 antagonist is Maraviroc. Maraviroc (brand-named Selzentry, or Celsentri outside the U.S.) is a chemokine receptor antagonist drug developed by the drug company Pfizer to act against HIV by interfering with the interaction between HIV and CCR5. It was approved for use by the FDA in August, 2007. In one example, Maraviroc blocks the activation of CCR5 that is expressed on the grafted NPCs by inflammatory cytokines like CCLs released by inflammatory cells infiltrating the neurological injury or disease site, reducing the apoptosis and promoting the differentiation/maturation of grafted NPCs. Thus, Maraviroc may effectively mitigate the inflammatory insult in a hostile inflammatory environment in the neurological injury or disease site such as an infarct core.

In one example, the Maraviroc in the composition as disclosed herein has a concentration of 3 mg/mL-50 mg/mL. In another example, the Maraviroc in the composition as disclosed herein has a concentration of about 3 mg/mL. In another example, the Maraviroc in the composition as disclosed herein has a concentration of about 5 mg/mL. In another example, the Maraviroc in the composition as disclosed herein has a concentration of about 10 mg/mL. In another example, the Maraviroc in the composition as disclosed herein has a concentration of about 15 mg/mL. In another example, the Maraviroc in the composition as disclosed herein has a concentration of about 20 mg/mL. In another example, the Maraviroc in the composition as disclosed herein has a concentration of about 25 mg/mL. In another example, the Maraviroc in the composition as disclosed herein has a concentration of about 30 mg/mL. In another example, the Maraviroc in the composition as disclosed herein has a concentration of about 35 mg/mL. In another example, the Maraviroc in the composition as disclosed herein has a concentration of about 40 mg/mL. In another example, the Maraviroc in the composition as disclosed herein has a concentration of about 45 mg/mL. In another example, the Maraviroc in the composition as disclosed herein has a concentration of about 50 mg/mL.

In another example, the CCR5 antagonist is a small molecule selected from the groups consisting of Fuscin, TAK-220, Nifeviroc, DAPTA, Aplaviroc, Aplaviroc hydrochloride, Ophiobolin C, AZD-5672, and Maraviroc-d6.

In another example, the CCR5 antagonist is a nucleic acid. In another example, the CCR5 antagonist is a small interfering RNA (siRNA). Small interfering RNA (siRNA) are typically double-stranded RNA molecules, 20-25 nucleotides in length. When transfected into cells, siRNA inhibit the target mRNA transiently until they are also degraded within the cell. In another example, the CCR5 antagonist is a Small hairpin RNA (shRNA). Small hairpin RNAs (shRNA) are sequences of RNA, typically about 80 base pairs in length, that include a region of internal hybridization that creates a hairpin structure. shRNA molecules are processed within the cell to form siRNA which in turn knock down gene expression. In another example, the CCR5 antagonist is a micro-RNA (miRNA). miRNAs are small non-coding RNAs, with an average 22 nucleotides in length. miRNAs are partially complementary to one or more messenger RNA (mRNA) molecules, and they can downregulate gene expression in a variety of manners, including translational repression, mRNA cleavage, and deadenylation.

In one example, the siRNA in the composition as disclosed herein has a concentration of 1×10⁶-1×10⁸ U/mL. In another example, the shRNA in the composition as disclosed herein has a concentration of 1×10⁶-1×10⁸ U/mL. In another example, the miRNA in the composition as disclosed herein has a concentration of 1×10⁶-1×10⁸ U/mL. In another example, the siRNA or shRNA or miRNA in the composition as disclosed herein has a concentration of about 1×10⁶ U/mL. In another example, the siRNA or shRNA or miRNA in the composition as disclosed herein has a concentration of about 5×10⁶ U/mL. In another example, the siRNA or shRNA or miRNA in the composition as disclosed herein has a concentration of about 1×10⁷ U/mL. In another example, the siRNA or shRNA or miRNA in the composition as disclosed herein has a concentration of about 5×10⁷ U/mL. In another example, the siRNA or shRNA or miRNA in the composition as disclosed herein has a concentration of about 1×10⁸ U/mL.

In one example, the nucleic acid based CCR5 antagonist is delivered to NPCs using a viral vector. In another example, the viral vector is a lentiviral vector. In another example, the viral vector is an adenoviral vector. In another example, the viral vector is an adeno-associated viral (AAV) vector. In another example, the viral vector is a retroviral vector. Advantageously, viral vectors usually give high transfection efficiencies. In another example, the nucleic acid based CCR5 antagonist is delivered to NPCs using a non-viral vector. In one example, the non-viral vector is an inorganic material-based vector selected from the group consisting of gold nanoparticles (AuNPs), mesoporous silicon, graphene oxide and Fe₃O₄-mediated nanoparticles (NPs). In another example, the non-viral vector is a lipid-based nanocarrier selected from the group consisting of a cationic lipid such as Lipofectamine, and a neutral lipid such as cholesterol, dioleylphosphatidyl choline (DOPC) and dioleylphosphatidyl ethanolamine (DOPE). In another example, the non-viral vector is a polymeric vector selected from the group consisting of polyethylenimines (PEI), poly(lactide-co-glycolide) (PLGA), chitosan, and β-cyclodextrin. In another example, the non-viral vector is a dendrimer-based vector, such as PAMAM dendrimer.

In another example, the CCR5 antagonist is an antibody. In another example, the CCR5 antagonist is an antibody PRO 140 (Leronlimab).

As used herein, the term “neural precursor cells (NPCs)” refers to a mixed population of cells consisting of all undifferentiated progeny of neural stem cells (NSCs), therefore including both NSCs and neural progenitor cells. The term “neural precursor cells (NPCs)” is commonly used to collectively describe the mixed population of NSCs and neural progenitor cells.

As used herein, the term “neural stem cells (NSCs)” refer to multipotent cells of the central nervous system (CNS, made up of the brain and spinal cord) which are able to self-renew and proliferate without limit, and to produce progeny cells which terminally differentiate into many, if not all, of the glial and neuronal cell types that populate the CNS, such as neurons, or glial cells including astrocytes and oligodendrocytes. The non-stem cell progeny of NSCs are referred to as neural progenitor cells.

As used herein, the term “neural progenitor cells” refer to cells which have the capacity to proliferate and differentiate into at least one cell type. Neural progenitor cells can therefore be unipotent, bipotent or multipotent. A distinguishing feature of a neural progenitor cell is that, unlike a stem cell, it has a limited proliferative ability and does not exhibit self-renewal.

In one example, NPCs are generated in vitro by differentiating embryonic stem cells (ESCs). In another example, NPCs are generated in vitro by differentiating induced pluripotent stem cells (iPSCs). iPSCs are derived from adult cells, most often from fibroblasts or blood cells, and programmed into an embryonic-like pluripotent state.

In one example, NPCs are embryonic NPCs, isolated from the CNS of developing embryos. During mammalian CNS development, NPCs arising from the neural tube produce pools of multipotent and more restricted neural progenitor cells, which then proliferate, migrate and further differentiate into neurons and glial cells. During embryogenesis, NPCs are derived from the neuroectoderm and can first be detected during neural plate and neural tube formation. As the embryo develops, NSCs can be identified in nearly all regions of the embryonic CNS, including the septum, cortex, thalamus, ventral mesencephalon and spinal cord. NSCs isolated from these regions have a distinct spatial identity and differentiation potential.

In another example, NPCs are adult NPCs, isolated from the CNS of mature adults. In another example, adult NPCs are found in the regions of the CNS of mature adults selected from the group consisting of the subgranular zone in the hippocampal dentate gyrus, the subventricular zone around the lateral ventricles, and the hypothalamus (precisely in the dorsal α1, α2 region and the “hypothalamic proliferative region”, located in the adjacent median eminence).

The NPCs may be grafted or transplanted into a neurological injury or disease site together with the composition as disclosed herein, which supports survival and differentiation of the grafted NPCs. As used herein, the term “neurological injury or disease site” refers to a site resulted from a neurological injury or disease characterized by inflammation, selected from the group consisting of stroke, traumatic brain injury, spinal cord injury, multiple sclerosis, Alzheimer's disease, Parkinson's disease, Huntington disease, Amyotrophic lateral sclerosis (ALS), epilepsy, hypoxia-induced nerve cell damage as in cardiac arrest or neonatal distress, neurological conditions associated with cancer, and neurodegenerative disease. In one example, the neurological injury or disease site results from stroke, specifically a cerebral stroke. A cerebral stroke is a sudden and permanent death of brain cells that occurs when the flow of blood is blocked and oxygen cannot be delivered to the brain. In one example, the cerebral stroke is an ischemic stroke. Ischaemic stroke most commonly occurs when the flow of blood is prevented by clotting (known as ‘thrombosis’ of the artery) or by a detached clot that lodges in an artery (referred to as an ‘embolic stroke’). In another example, the cerebral stroke is a haemorrhagic stroke. Haemorrhagic stroke results from rupture of an artery wall, and from blood leaking into the surrounding brain. Haemorrhagic stroke, like ischemic stroke, is a cause of death of tissue by depriving the brain of blood and oxygen, and results in a number of neurological disabilities (motor, speech) as well as functional disabilities. In another example, the neurological injury or disease site results from traumatic brain injury. Traumatic brain injury usually results from a violent blow or jolt to the head or body. An object that goes through brain tissue, such as a bullet or shattered piece of skull, also can cause traumatic brain injury. Traumatic brain injury can result in bruising, torn tissues, bleeding and other physical damage to the brain. These injuries can result in long-term complications or death. In one example, the traumatic brain injury is a closed brain injury. Closed brain injuries happen when there is a non-penetrating injury to the brain with no break in the skull, caused by a rapid forward or backward movement and shaking of the brain inside the bony skull that results in bruising and tearing of brain tissue and blood vessels. In another example, the traumatic brain injury is a penetrating brain injury. Penetrating, or open head injuries happen when there is a break in the skull, such as when a bullet pierces the brain. In another example, the neurological injury or disease site results from spinal cord injury. A spinal cord injury refers to damage to any part of the spinal cord or nerves at the end of the spinal canal (cauda equina), which often causes permanent changes in strength, sensation and other body functions below the site of the injury. In another example, the neurological injury or disease site results from multiple sclerosis (MS). In MS, the immune system attacks the protective sheath (myelin) that covers nerve fibers and may cause permanent damage or deterioration of the nerves. In another example, the neurological injury or disease site results from Alzheimer's disease, which is characterized by damaged nerve cells. In another example, the neurological injury or disease site results from Parkinson's disease. In Parkinson's disease, nerve cells in the basal ganglia, an area of the brain that controls movement, become impaired and/or die. In another example, the neurological injury or disease site results from Huntington disease, which causes movement, cognitive and psychiatric disorders with a wide spectrum of signs and symptoms. In another example, the neurological injury or disease site results from Amyotrophic lateral sclerosis (ALS). ALS affects the nerve cells that control voluntary muscle movements such as walking and talking (motor neurons). ALS causes the motor neurons to gradually deteriorate, and then die.

In one example, the neurological injury or disease site is at the brain. In another example, the neurological injury or disease site is at the spinal cord. In another example, the neurological injury or disease site is at the cerebrum. In another example, the neurological injury or disease site is at the cerebellum. In another example, the neurological injury or disease site is at the brainstem. In another example, the neurological injury or disease site is at frontal lobe of the cerebrum. In another example, the neurological injury or disease site is at the parietal lobe of the cerebrum. In another example, the neurological injury or disease site is at the occipital lobe of the cerebrum. In another example, the neurological injury or disease site is at the temporal lobe of the cerebrum. In another example, the neurological injury or disease site is at the midbrain of the brainstem. In another example, the neurological injury or disease site is at the pons of the brainstem. In another example, the neurological injury or disease site is at the medulla. In another example, the neurological injury or disease site is at two or more of the sites as described above. In another example, the neurological injury or disease site is located in a brain region selected from the group consisting of the pituitary gland, the hypothalamus, the amygdala, the hippocampus, the pineal gland, the ventricles and cerebrospinal fluid. In another example, the neurological injury or disease site have damaged cranial nerves selected from the group consisting of olfactory nerve, optic nerve, oculomotor nerve, trochlear nerve, trigeminal nerve, abducens nerve, facial nerve, vestibulocochlear nerve, glossopharyngeal nerve, vagus nerve, accessory nerve, and hypoglossal nerve. In another example, the neurological injury or disease site have damaged blood vessels selected from the group consisting of the basilar artery, the vertebral arteries, the external carotid arteries, the internal carotid arteries, and the circle of Willis. In another example, the neurological injury or disease site is focal (confined to one area of the brain). In another example, the neurological injury or disease site is diffuse (happens in more than one area of the brain).

In animals with a neurological injury or disease as disclosed herein, such as ischemic stroke, the neurological injury or disease site has deceased neural cells, or damaged blood vessels, or both. In one example, the ischemic/infarct site forms a cavity that is walled off by glial scars and filled with inflammatory cells and secretions. Such an inflammatory environment is hostile to transplanted NPCs, allowing few transplanted NPCs to survive. The composition disclosed herein supports the survival and differentiation of transplanted NPCs at the neurological injury or disease site as disclosed herein.

In one example, the composition comprising the gel forming molecule and the CCR5 antagonist as disclosed herein further comprises calcium. In another example, the composition comprising fibrinogen and Maraviroc further comprises CaCl₂). Advantageously, calcium decreases the time required for fibrin formation from fibrinogen by markedly accelerating the phase of fibrin monomer polymerization and gel formation. The gel effectively holds the grafted NPCs together and prevents the rapid dilution and/or degradation of Maraviroc, supporting the survival of transplanted NPCs. In one example, the CaCl₂) in the composition as disclosed herein has a concentration of 1-5 mM. In a particular example, the CaCl₂) in the composition as disclosed herein has a concentration of 2.5 mM.

In another example, the composition comprising the gel forming molecule and the CCR5 antagonist as disclosed herein does not comprise calcium. After administering the mixture of the NPCs and the composition as disclosed herein to the neurological injury or disease site, calcium present in the body fluid will promote gel formation.

In another example, the composition comprising the gel forming molecule and the CCR5 antagonist as disclosed herein further comprises thrombin. Thrombin mediates proteolytic cleavage and removal of N-terminal fibrinopeptides from the Aα and Bβ chains of fibrinogen and leads to fibrin (gel) formation. In one example, the thrombin in the composition as disclosed herein has a concentration of 10 U/mL-500 U/mL. In another example, the thrombin in the composition as disclosed herein has a concentration of about 10 U/mL. In another example, the thrombin in the composition as disclosed herein has a concentration of about 50 U/mL. In another example, the thrombin in the composition as disclosed herein has a concentration of about 100 U/mL. In another example, the thrombin in the composition as disclosed herein has a concentration of about 200 U/mL. In another example, the thrombin in the composition as disclosed herein has a concentration of about 300 U/mL. In another example, the thrombin in the composition as disclosed herein has a concentration of about 400 U/mL. In another example, the thrombin in the composition as disclosed herein has a concentration of about 500 U/mL.

In another example, the composition comprising the gel forming molecule and the CCR5 antagonist as disclosed herein does not comprise thrombin. After administering the mixture of the NPCs and the composition as disclosed herein to the neurological injury or disease site, thrombin present in the body fluid will facilitate gel formation.

In another example, the composition as disclosed herein further comprises a growth factor selected from the group consisting of brain-derived neurotrophic factor (BDNF), nerve growth factor (NGF), neurotrophin, platelet-derived growth factor (PDGF), glial cell line-derived neurotrophic factor (GDNF), insulin-like growth factor-1 (IGF-1), insulin-like growth factor-2 (IGF-2), fibroblast growth factor (FGF), vascular endothelial growth factor (VEGF), and bone morphogenetic proteins (BMP4). Growth factors may be used in the composition as disclosed herein depending on the nature of the NPCs, the number of NPCs to be transplanted, and the size of the lesion to be filled. In one example, the growth factors disclosed herein are included in the composition if the neurological injury or disease site such as a lesion is large, and cells need to divide more to fill it. In another example, the brain-derived neurotrophic factor (BDNF) in the composition has a concentration of 1 ng/mL-100 ng/mL. In another example, the nerve growth factor (NGF) in the composition has a concentration of 1 ng/mL-100 ng/mL. In another example, the neurotrophin in the composition has a concentration of 1 ng/mL-50 ng/mL. In another example, the platelet-derived growth factor (PDGF) in the composition has a concentration of 1 ng/mL-100 ng/mL. In another example, the glial cell line-derived neurotrophic factor (GDNF) in the composition has a concentration of 1 ng/mL-500 ng/mL. In another example, the insulin-like growth factor-1 (IGF-1) in the composition has a concentration of 1 ng/mL-50 ng/mL. In another example, the insulin-like growth factor-2 (IGF-2) in the composition has a concentration of 1 ng/mL-50 ng/mL. In another example, the fibroblast growth factor (FGF) in the composition has a concentration of 1 ng/mL-200 ng/mL. In another example, the vascular endothelial growth factor (VEGF) in the composition has a concentration of 10 ng/mL-500 ng/mL. In another example, the bone morphogenetic proteins (BMP4) in the composition has a concentration of 5 ng/mL-500 ng/mL. Advantageously, the concentrations of growth factors in the composition as disclosed herein are similar to their physiological concentrations. This avoids the problem of the microgram level growth factor conventionally used in the field with fibrinogen to support grafted cells in the injury site, i.e., conventionally growth factors used have over 1000 folds of the physiological concentrations. At such high concentrations, these growth factors promote the proliferation of the transplanted NPCs, forming “space-occupying” tissues like tumors in the spinal cord, which may cause secondary injury by compressing the intact region. The use of a high concentration (over 1000 folds of the physiological concentration) of growth factors also prevents NPCs differentiation. Hence, the transplanted NPCs in the conventional studies retained the neural precursor state for many months. In contrast, the concentrations of the growth factors used herein prevents the scenario wherein the grafted NPCs are kept in proliferation state leading to overgrowth into a “space-occupying” tissue.

In another example, the composition as disclosed herein does not comprise a growth factor selected from the group consisting of brain-derived neurotrophic factor (BDNF), nerve growth factor (NGF), neurotrophin, platelet-derived growth factor (PDGF), glial cell line-derived neurotrophic factor (GDNF), insulin-like growth factor-1 (IGF-1), insulin-like growth factor-2 (IGF-2), fibroblast growth factor (FGF), vascular endothelial growth factor (VEGF), and bone morphogenetic proteins (BMP4). This prevents overgrowth of grafted NPCs in the case of a relatively small lesion.

In another aspect, the present disclosure refers to a method of treating a neurological injury or disease of a subject, comprising (a) mixing NPCs with the composition as disclosed herein; and (b) administering a mixture of the NPCs and the composition into a neurological injury or disease site of the subject, to thereby support survival and differentiation of the NPCs.

In another aspect, the present disclosure refers to use of a mixture of NPCs and the composition as disclosed herein in the manufacture of a medicament for treating a neurological injury or disease of a subject, wherein the mixture is to be administered into a neurological injury or disease site of the subject, to thereby support survival and differentiation of the NPCs.

As used herein, the term “treating” and grammatical variations of that term, refer to administration of a mixture of the NPCs and the composition as disclosed herein to a subject as described herein by any appropriate means as described herein. Such treatment includes any and all uses which remedy a disease state or symptoms, prevent the establishment of disease, or otherwise prevent, hinder, retard, or reverse the progression of disease or other undesirable symptoms in any way whatsoever.

As disclosed herein, the term “neural precursor cells (NPCs)” is used to collectively describe the mixed population of NSCs and neural progenitor cells. In one example, NPCs are generated in vitro by differentiating embryonic stem cells (ESCs). In another example, NPCs are generated in vitro by differentiating induced pluripotent stem cells (iPSCs). iPSCs are derived from adult cells, most often from fibroblasts or blood cells, and programmed into an embryonic-like pluripotent state. In one example, NPCs are embryonic NPCs, isolated from the CNS of developing embryos. During mammalian CNS development, NPCs arising from the neural tube produce pools of multipotent and more restricted neural progenitor cells, which then proliferate, migrate and further differentiate into neurons and glial cells. During embryogenesis, NPCs are derived from the neuroectoderm and can first be detected during neural plate and neural tube formation. As the embryo develops, NSCs can be identified in nearly all regions of the embryonic CNS, including the septum, cortex, thalamus, ventral mesencephalon and spinal cord. NSCs isolated from these regions have a distinct spatial identity and differentiation potential. In another example, NPCs are adult NPCs, isolated from the CNS of mature adults. In another example, adult NPCs are found in the regions of the CNS of mature adults selected from the group consisting of the subgranular zone in the hippocampal dentate gyrus, the subventricular zone around the lateral ventricles, and the hypothalamus (precisely in the dorsal α1, α2 region and the “hypothalamic proliferative region”, located in the adjacent median eminence).

In one example, the neurological injury or disease as disclosed herein is a disease or disorder of the central nervous system including, but not limited to, stroke, traumatic brain injury, spinal cord injury, multiple sclerosis, Alzheimer's disease, Parkinson's disease, Huntington disease, Amyotrophic lateral sclerosis (ALS), epilepsy, hypoxia-induced nerve cell damage as in cardiac arrest or neonatal distress, neurological conditions associated with cancer, and neurodegenerative disease. In one example, the neurological injury or disease is stroke, specifically a cerebral stroke. A cerebral stroke is a sudden and permanent death of brain cells that occurs when the flow of blood is blocked and oxygen cannot be delivered to the brain. In one example, the cerebral stroke is an ischemic stroke. Ischaemic stroke most commonly occurs when the flow of blood is prevented by clotting (known as ‘thrombosis’ of the artery) or by a detached clot that lodges in an artery (referred to as an ‘embolic stroke’). In another example, the cerebral stroke is a haemorrhagic stroke. Haemorrhagic stroke results from rupture of an artery wall, and from blood leaking into the surrounding brain. Haemorrhagic stroke, like ischemic stroke, is a cause of death of tissue by depriving the brain of blood and oxygen, and results in a number of neurological disabilities (motor, speech) as well as functional disabilities. In another example, the neurological injury or disease is traumatic brain injury. Traumatic brain injury usually results from a violent blow or jolt to the head or body. An object that goes through brain tissue, such as a bullet or shattered piece of skull, also can cause traumatic brain injury. Traumatic brain injury can result in bruising, torn tissues, bleeding and other physical damage to the brain. These injuries can result in long-term complications or death. In one example, the traumatic brain injury is a closed brain injury. Closed brain injuries happen when there is a non-penetrating injury to the brain with no break in the skull, caused by a rapid forward or backward movement and shaking of the brain inside the bony skull that results in bruising and tearing of brain tissue and blood vessels. In another example, the traumatic brain injury is a penetrating brain injury. Penetrating, or open head injuries happen when there is a break in the skull, such as when a bullet pierces the brain. In another example, the neurological injury or disease is spinal cord injury. A spinal cord injury refers to damage to any part of the spinal cord or nerves at the end of the spinal canal (cauda equina), which often causes permanent changes in strength, sensation and other body functions below the site of the injury. In another example, the neurological injury or disease is multiple sclerosis (MS). In MS, the immune system attacks the protective sheath (myelin) that covers nerve fibers and may cause permanent damage or deterioration of the nerves. In another example, the neurological injury or disease is Alzheimer's disease, which is characterized by damaged nerve cells. In another example, the neurological injury or disease is Parkinson's disease. In Parkinson's disease, nerve cells in the basal ganglia, an area of the brain that controls movement, become impaired and/or die. In another example, the neurological injury or disease is Huntington disease, which causes movement, cognitive and psychiatric disorders with a wide spectrum of signs and symptoms. In another example, the neurological injury or disease is Amyotrophic lateral sclerosis (ALS). ALS affects the nerve cells that control voluntary muscle movements such as walking and talking (motor neurons). ALS causes the motor neurons to gradually deteriorate, and then die.

The terms “subject”, “host”, and “patient” are used interchangeably. As used herein, a subject is preferably a mammal, such as a non-primate (e.g., cows, pigs, horses, cats, dogs, rats, etc.) or a primate (e.g., monkeys and humans), most preferably a human.

In one example, the number of NPCs to be mixed with the composition as disclosed herein and to be administered into a neurological injury or disease site is 50,000-500,000. In another example, the number of NPCs to be mixed with the composition as disclosed herein and to be administered into a neurological injury or disease site is 50,000-100,000. In another example, the number of NPCs to be mixed with the composition as disclosed herein and to be administered into a neurological injury or disease site is 100,000-200,000. In another example, the number of NPCs to be mixed with the composition as disclosed herein and to be administered into a neurological injury or disease site is 200,000-300,000. In another example, the number of NPCs to be mixed with the composition as disclosed herein and to be administered into a neurological injury or disease site is 300,000-400,000. In another example, the number of NPCs to be mixed with the composition as disclosed herein and to be administered into a neurological injury or disease site is 400,000-500,000. In another example, the number of NPCs to be mixed with the composition as disclosed herein and to be administered into a neurological injury or disease site is about 50,000. In another example, the number of NPCs to be mixed with the composition as disclosed herein and to be administered into a neurological injury or disease site is about 100,000. In another example, the number of NPCs to be mixed with the composition as disclosed herein and to be administered into a neurological injury or disease site is about 200,000. In another example, the number of NPCs to be mixed with the composition as disclosed herein and to be administered into a neurological injury or disease site is about 300,000. In another example, the number of NPCs to be mixed with the composition as disclosed herein and to be administered into a neurological injury or disease site is about 400,000. In another example, the number of NPCs to be mixed with the composition as disclosed herein and to be administered into a neurological injury or disease site is about 500,000. In another example, the number of NPCs to be mixed with the composition as disclosed herein and to be administered into a neurological injury or disease site is more than 500,000.

In one example, the NPCs are mixed with the composition comprising the gel forming molecule, the CCR5 antagonist and thrombin as disclosed herein, and administered to a neurological injury or disease site after mixing. In another example, the NPCs are mixed with the composition comprising the gel forming molecule and the CCR5 antagonist as disclosed herein, and administered to a neurological injury or disease site, followed by administering thrombin into the neurological injury or disease site. Thrombin may be added separately and subsequently to the administration of the mixture of the NPCs and the composition comprising the gel forming molecule and the CCR5 antagonist as disclosed herein, to avoid gel formation prematurely before transplantation.

As used herein, the term “administration” and grammatical variations of that term, refer to transplanting the mixture of NPCs and the composition as disclosed herein to a neurological injury or disease site of the subject as disclosed herein. In one example, the mixture of NPCs and the composition as disclosed herein is administered by stereotaxic injection, which allows injecting NPCs directly into the neurological injury or disease site as disclosed herein.

Cell therapy for neurological injuries and diseases such as stroke, traumatic brain injury, spinal cord injury, multiple sclerosis, Alzheimer's disease, Parkinson's disease, Huntington disease, Amyotrophic lateral sclerosis (ALS), epilepsy, hypoxia-induced nerve cell damage as in cardiac arrest or neonatal distress, neurological conditions associated with cancer, and neurodegenerative disease require coordinated and dynamic regulation of transplanted NPCs, including maximum survival, appropriate degree of proliferation, neuronal vs. glial differentiation, and synaptogenesis. Advantageously, the composition as disclosed herein when used in combination with NPCs, supports NPCs survival and differentiation to mature neurons after the NPCs being transplanted to a neurological injury or disease site. Due to the high level of CCR5 found to be expressed on NPCs, the CCR5 antagonist promotes NPCs survival via blocking CCR5 signaling. In addition, the gel forming molecule forms a gel, which holds the grafted NPCs together and prevents the grafted NPCs from “swimming” in the injury cyst or cavity. In the presence of fibrinogen and Maraviroc, the grafted NPCs are evenly distributed without clustering and differentiate to mature neurons at about 30 days after transplantation.

In another aspect, the present disclosure refers to a kit for use in supporting survival and differentiation of NPCs grafted into a neurological injury or disease site, the kit comprising: (a) the composition as disclosed herein; (b) artificial cerebral spinal fluid (a-CSF); (c) CaCl₂); and (d) thrombin.

In one example, the kit further comprises carriers, diluents and adjuvants for administering the mixture of NPCs and the composition as disclosed herein. The carriers, diluents and adjuvants must be pharmaceutically “acceptable” in terms of being compatible with the other ingredients of the composition, and not deleterious to the recipient thereof.

Examples of pharmaceutically acceptable carriers or diluents are demineralized or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrolidone; agar; gum tragacanth or gum acacia, and petroleum jelly. Typically, the carrier or carriers will form from 10% to 99.9% by weight of the compositions.

As used in this application, the singular form “a,” “an,” and “the” include plural references unless the context clearly dictates otherwise. For example, the term “a primer” includes a plurality of primers, including mixtures and combinations thereof.

As used herein, the term “comprising” means “including.” Variations of the word “comprising”, such as “comprise” and “comprises,” have correspondingly varied meanings. Thus, for example, a composition “comprising” X may consist exclusively of X or may include one or more additional unrecited components.

As used herein, the term “about” in the context of concentration of a substance, size of a substance, length of time, or other stated values means+/−5% of the stated value, or +/−4% of the stated value, or +/−3% of the stated value, or +/−2% of the stated value, or +/−1% of the stated value, or +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Unless otherwise indicated, all technical and scientific terms used herein have the same meaning as commonly understood to one of ordinary skill in the art to which the invention belongs.

Other embodiments are within the following claims and non-limiting examples.

### EXAMPLES

Non-limiting examples of the disclosure will be further described in greater detail by reference to specific Examples, which should not be construed as in any way limiting the scope of the disclosure.

### Example 1—Methods

### Cell Culture

Human embryonic stem cells (ESCs, eGFP H9 hESC line) were cultured in feeder-free mTeSR medium with 1×mTeSR supplement, 1×Non-Essential Amino Acids (NEAA) and 1×Glutamax in a Matrigel-coated 6-well plate. ESCs were fed daily and passaged twice a week.

Induction of forebrain glutamate NPCs was done using the following procedure. Briefly, H9 hESCs or eGFP H9 hESCs were cultured on 6-well plates with Matrigel/vitronectin coated for one week. ESC colonies were gently blown off by 1 mL pipettes to form cell aggregates at day 0. Cell aggregates were cultured in flasks for 7 days with the neural induction medium (NIM) consisting of DMEM/F12, 1×N2 supplement, 1×NEAA, 2-μM SB431542, and 2-μM DMH-1. Cell aggregates were adhered to 6-well plates in the presence of NIM with 5% FBS for 6 hours, and then fresh NIM was changed. The aggregates were fed with NIM till neural rosette formation at day 16. The rosettes were gently blown off using 1 mL pipettes and suspended in flasks with NIM for 7 days. Then, NIM was changed every four days from day 23. The neural precursor cells (NPCs) were maintained in NIM till transplantation or immunostaining. NPCs were digested into single cells using TrypLE for 3 min at day 49. After one additional day of culture in NIM supplemented with 1×B27 and 100 nM compound E, the NPCs were collected for transplantation in ischemic stroke models. B-27 supplement is a defined yet complex mixture of antioxidant enzymes, proteins, vitamins, and fatty acids that are combined in optimized ratios to support neuronal survival in culture. Compound E is a γ-secretase inhibitor. For immunostaining, NPCs were seeded on glass coverslips and staining was performed after one-week culture.

To test the effects of CCR5 activation on NPCs and neurons, cells were seeded on Matrigel coated coverslips or 6-well plates and treated with overdose (300 ng/mL) ligands of CCR5. CCL3, CCL4, CCL5 and their combination (CCL3/4/5) were respectively added into NIM every other day. After four-day treatment, NPCs or neurons were collected for staining or immunoblotting.

### Lentivirus Production

Human CCR5 29mer shRNA plasmids (pRS_hU6_CCR5shRNA_SV40_Puro) and Non-effective 29-mer scrambled shRNA cassette in pRS Vector were obtained from OriGene (CAT #: TR314126, CAT #: TR30012). The lentiviral shRNAs were generated in HEK 293FT cell line by transfecting packaging and backbone plasmids. HEK 293FT cells were cultured in DMEM with 10% FBS. The supernatant was collected after 3-day culture. Viral particles were concentrated by ultracentrifugation at 25000 rpm for 2.5 hours at 4° C. The viral particles were resuspended in DMEM.

Transduction of shRNA

1×10^{\hat{ }5} NPCs were seeded on coverslips in each well of 24-well plate for two days to 50% confluency upon transduction at 37° C. in a humidified 5% C02 incubator. The lentiviral shRNAs (MOIs of 20) were used to infect NPCs overnight at 37° C. The medium containing lentiviral particles was removed from wells and replaced with 500 L fresh pre-warmed NIM. The infected NPCs were collected for immunostaining or western blotting at 5 days after transduction.

### Preparation of Cocktail/Composition

Stock solution of 30 mg/mL fibrinogen, 50 mg/mL maraviroc and 250 mM CaCl₂) (100×) were prepared in the following manner: fibrinogen (F3879, Sigma) was dissolved in a-CSF for 1 hour at room temperature. Maraviroc was dissolved in Dimethyl Sulfoxide (DMSO). CaCl₂) (Sigma) was dissolved in deionized (DI) water. All solutions were sterile filtered and stored at −20° C. for use. Cocktail/composition was made of 50 mg/mL Maraviroc and 30 mg/mL fibrinogen in a volume ratio of 1:9 with 2.5 mM CaCl₂).

Release Profile of Maraviroc within Cocktail Gel

The wavelength of maximum absorbance (λₘₐₓ) of maraviroc in Phosphate buffer (pH7.4) was found 210 nm by scanning them over the UV range of 2000 nm to 400 nm. Standard drug solution of maraviroc was prepared by dissolving 50 mg pure Maraviroc in phosphate buffer 7.4 and transferred into 5 mL volumetric flask to obtain 10 mg/mL of stock solution and the resulting Maraviroc. Solution was used as working standard solution from which desired concentrations of solution were prepared. The final concentration of 0, 0.5, 1.0, 1.5, 2.0, 2.5, 3.0, 3.5, 4.0, 4.5, 5.0, 5.5, 6.0 mg/mL and absorbances were taken at λₘₐₓ 210 nm using an appropriate blank. Inducing the gelation of 200 μL fibrinogen (9 mg/mL) by thrombin (50 U) in vitro, and the gel with 1 mg Maraviroc was incubated in 1 mL PBS with pH=7.4 at 37° C. For free drug group, 5 mg maraviroc was directly dissolved in 1 mL PBS. Fifty microliters of solution was taken to test the absorbance by Microplate Absorbance Spectrophotometer (Bio-RAD, xMark™). The final concentrations of Maraviroc in PBS were calculated according to calibration curve.

### Stroke Model and Cell Transplantation

All animal studies were performed in accordance with the institutional animal care and use committee at Duke-NUS Medical School. Ischemic strokes in adult (10-12 weeks) male SCID mice were induced through photo-thrombosis. Briefly, Rose Bengal was administered intravenously at a dose of 0.1 mg/g per mouse. Mouse skull was exposed under 2% isoflurane anaesthesia. The right motor cortex (anterior-posterior [AP]=+2 mm, lateral [L]=+1 mm) received a 2.5-mm diameter illumination of cold light through the intact skull for 15 min. Animals were randomly grouped and transplanted with forebrain glutamatergic precursors. Fifty thousand cells were resuspended in 1 μl artificial cerebral spinal fluid (a-CSF) in the presence or absence of Maraviroc (5 mg/mL) or fibrinogen (10 mg/mL) and injected into the injured site ([AP]=+2 mm, [L]=+1 mm, vertical [V]=−1.5 mm, from dura).

### Tissue Preparation and Immunohistochemistry

Animals were sacrificed with a lethal dose of pentobarbital (250 mg/kg) and immediately perfused with PBS followed by 4% cold paraformaldehyde (PFA). The brain samples were fixed in cold PFA for 2 hours and immersed in 30% sucrose at 4° C. for around 4 days until sunk. Serial coronal (1.54 mm to-0.22 mm from Bregma) sections were collected on a freezing microtome at a 40-μm thickness and stored at −20° C. For immunostaining, sections were incubated with blocking solution containing 10% normal donkey serum and 0.2% Triton-100 for 1 h at room temperature. Then sections were incubated with primary antibodies overnight at 4° C.

| Antibodies | SOURCE | IDENTIFIER | WEB LINK |
|---|---|---|---|
| anti-GFAP | Cell | 34001 | https://www.cellsignal.com/products/ |
| | Signaling | | primary-antibodies/gfap-e6n91-mouse- |
| | Tech | | mab/34001?N=4294956287&Ntt= |
| | | | gfap&Nrpp=60&No=%7Boffset% |
| | | | 7D&fromPage=plp |
| anti-GFAP | Agilent | Z0334 | https://www.agilent.com/en/product/ |
| | Dako | | immunohistochemistry/antibodies- |
| | | | controls/primary-antibodies/glial- |
| | | | fibrillary-acidic-protein- |
| | | | (concentrate)-76683 |
| anti-SOX2 | R&D Systems | AF2018-SP | https://www.rndsystems.com/products/ |
| | | | human-mouse-rat-sox2-antibody- |
| | | | 245610_mab2018 |
| anti-DCX | Cell | 4604 | https://www.cellsignal.com/products/ |
| | Signaling | | primary-antibodies/doublecortin- |
| | Tech | | antibody/4604 |
| anti-Cleaved- | Cell | 9661S | https://www.cellsignal.com/products/ |
| caspase3 | Signaling | | primary-antibodies/cleaved- |
| | Tech | | caspase-3-asp175-antibody/9661 |
| anti- | Takara | Y40410 | https://www.takarabio.com/products/ |
| STEM121 | | | antibodies-and-elisa/primary- |
| | | | antibodies-and-elisas-by-research- |
| | | | area/stem-cell-research-antibodies/ |
| | | | stem-antibodies?catalog=Y40410 |
| anti-NF-L | Abcam | ab7255 | https://www.abcam.com/68kda- |
| | | | neurofilamentnf-1-antibody-da2- |
| | | | ab7255.html |
| anti-NeuN | Merck | MAB377 | https://www.merckmillipore.com/ |
| | Millipore | | CN/zh/product/Anti-NeuN- |
| | | | Antibody-clone-A60, MM_NF-MAB377 |
| anti-CCR5 | Abcam | ab110103 | https://www.abcam.com/ccr5- |
| | | | antibody-t218-ab110103.html |
| anti-Ctip2 | Abcam | ab18465 | https://www.abcam.com/ctip2- |
| | | | antibody-25b6-ab18465.html |
| anti-Brn2 | Proteintech | 14596-1-AP | https://www.ptgcn.com/products/ |
| | | | POU3F2-Antibody-14596-1-AP.htm |
| anti-Foxp2 | Abcam | ab16046 | https://www.abcam.com/foxp2- |
| | | | antibody-ab16046.html |
| anti-Ki67 | Cell | 9129S | https://www.cellsignal.com/products/ |
| | Signaling | | primary-antibodies/ki-67-d3b5- |
| | Tech | | rabbit-mab/9129 |
| anti-Sox9 | R&D Systems | AF3075 | https://www.rndsystems.com/products/ |
| | | | human-sox9-antibody_af3075 |
| anti-S100β | Abcam | ab52642 | https://www.abcam.com/s100- |
| | | | beta-antibody-ep1576y-astrocyte- |
| | | | marker-ab52642.html |
| anti-CSPG | Abcam | ab11570 | https://www.abcam.com/chondroitin- |
| | | | sulfate-antibody-cs-56-ab11570.html |
| anti-Iba1 | Wako Pure | 019-19741 | https://labchem-wako.fujifilm.com/ |
| | Chemical | | us/product/detail/W01W0101-1974.html |
| | Corporation | | |
| anti-Laminin | Novusbio | NB300-144 | https://www.novusbio.com/products/ |
| | | | laminin-antibody_nb300-144 |
| anti-MAP2 | Cell | 8707S | https://www.cellsignal.com/products/ |
| | Signaling | | primary-antibodies/map2-d5g1-xp- |
| | Tech | | rabbit-mab/8707?applicationCode= |
| | | | IF&productId=3658&Ns=product.status. |
| | | | releasedDateIso%7C1&N=4294967218+ |
| | | | 4294956287&Nrpp=200&No=2600& |
| | | | fromPage=plp |

Sections were subsequently washed and incubated with corresponding secondary antibodies for 1 h at room temperature. Immunolabeled sections were mounted by Fluoromount-G with Hoechst. For TUNEL staining, cells on coverslips were fixed in PBS with 4% PFA for 30 mins, and then incubated with Terminal Deoxynucleotidyl Transferase (TdT) Equilibration buffer (Elabscience) at 37° C. for 30 mins. NPCs were incubated in labeling solution (Elabscience) with TdT enzyme at 37° C. for 1 hour.

### Imaging and Cellular Quantification

To quantify the population of DCX, SOX2, Ki67, NF and NeuN positive cells to total grafted cells (eGFP and Hoechst co-labelled), one brain slice was selected from every 6 serial slices on for stereological counting on Zeiss M1 Microscope with Stereo Investigator software (MBF Bioscience). In brief, immunolabeled slices were scanned on Zeiss M1 Microscope, grafts area was contoured manually, and then corresponding fluorescence labelled cells were unbiasedly counted. Population of cleaved-caspase3 expressing cells to total grafted cells was counted with ImageJ software. Data were replicated 4 to 6 times per group. To quantify the population of Brn2, Ctip2 and Foxp2 expressing cells to total GFP⁺ cells on coverslips, all coverslips were scanned and captured by 20× objective with a confocal microscope (Nikon), and then was counted with ImageJ software. Data were replicated three times. All data were expressed as means±SEM.

### Infarct Area Analysis and Quantification

The infarct area was defined by GFAP, S100β, CSPG and Iba1 staining. Fluorescence intensity in the infarct area was measured with ImageJ software and normalized to the surrounding intact region. To measure the thickness of infarct area, six slices were chosen randomly from 35 brain slices and captured by a confocal microscope (Nikon). The vertical distance from surface in epicenter to corpus callosum was measured with ImageJ software. All data were replicated four to six times and were expressed as means±SEM.

### Behavior Tests

Mice (n=9-10 per group) were tested on the rotarod and grid-walk tasks. Behavior was assessed −14, 0, 14 and 30 days following transplantation. For the rotarod test, latency of fall was calculated to assess the motor function. For the grid-walk test, deficit was calculated as the number of impaired limb (right foot) within 10 min.

### Isolation of Injured Tissue

Animals were anaesthetized and perfused with cold PBS at 2, 14 and 44 days after stroke. The injured cortices or grafts (an area with a radius of 1 mm from the epicenter) were dissected manually under a stereomicroscope (Zeiss) and stored at −80° C.

### Western Blotting

NPCs and neurons were washed using PBS and resuspended in RIPA buffer with protease inhibitor and phosphatase inhibitor. Samples were collected to 1.5 mL tubes on ice for 15 mins. For tissue samples, extracts were sonicated in cold RIPA buffer with protease/phosphatase inhibitors. All samples were quantified using Quick Start™ Bradford Protein Assay (Bio-RAD), and then Laemmli buffer (Bio-RAD) was added to each tube. Samples were heated at 95° C. for 5 mins and stored at −80° C. Total 15 μg of extract was loaded to each well on a 10% Bis-Tris pre-casted gel for electrophoresis.

After running at 120 V for 40 mins, proteins were transferred to PVDF membranes at 400 mA for 30 mins. Membranes were subsequently blocked in 0.1% TBS-Tween (TBST) with 5% non-fat milk for 1 hour. Then membranes were incubated with primary antibodies overnight at 4° C.: anti-CCR5 (1:1000, Abcam, ab110103), anti-CCL3 (1:1000, Abcam, ab259372), anti-CCL4 (1:1000, Abcam, EP521Y), anti-CCL5 (1:1000, Thermo Fisher Scientific, 701030), anti-GAPDH (1:5000, Thermo Fisher Scientific, MA5-15738). The membranes were washed three times by 0.1% TBST and incubated with corresponding secondary antibodies at room temperature for 1 hour. The protein bands were presented by Enhanced Chemiluminescence Substrate (Promega) and visualized in Bio-RAD Chemidoc system. All intensity of bands was analysed by ImageJ software and normalized to corresponding GAPDH bands.

### Quantification and Statistical Analysis

The data of the stroke group and the transplanted groups are normally distributed. Unpaired t-test was used for comparison between two groups. Error bars in all figures represent means±SEM. Differences were considered statistically significant at a P value of less than 0.05. All data were analyzed using GraphPad Prism.

### Example 2—Results

Combination of Fibrinogen and CCR5 Antagonist Protects Grafted NPCs from Apoptosis in the Ischemic Core

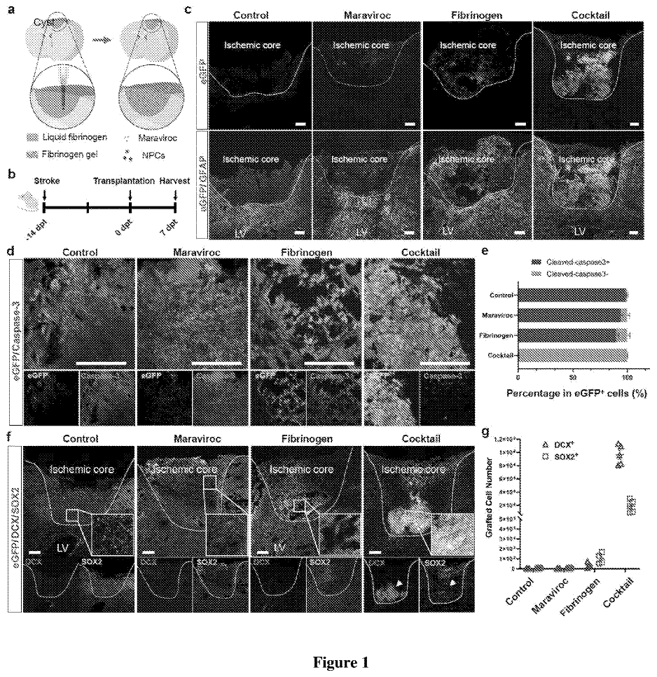

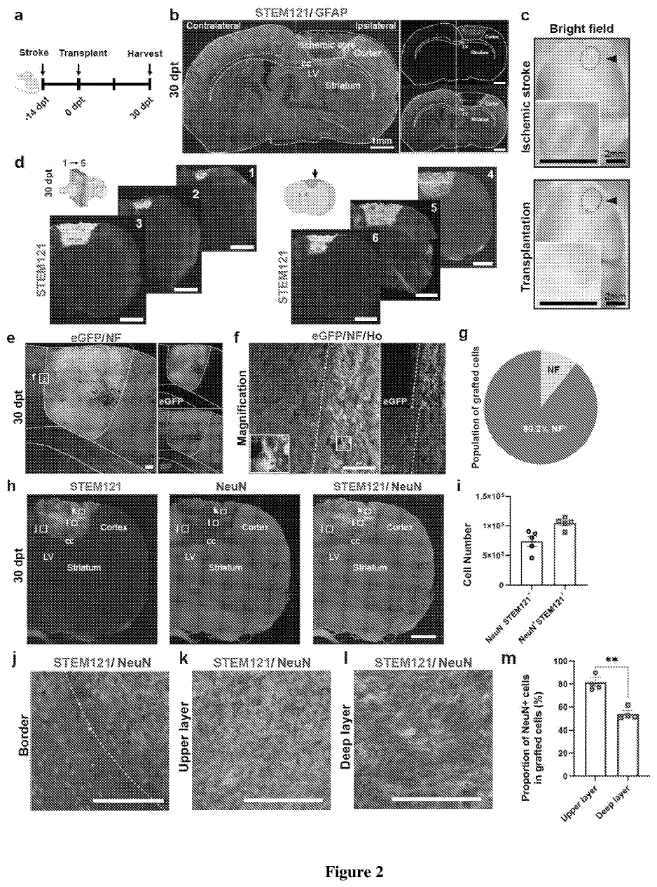

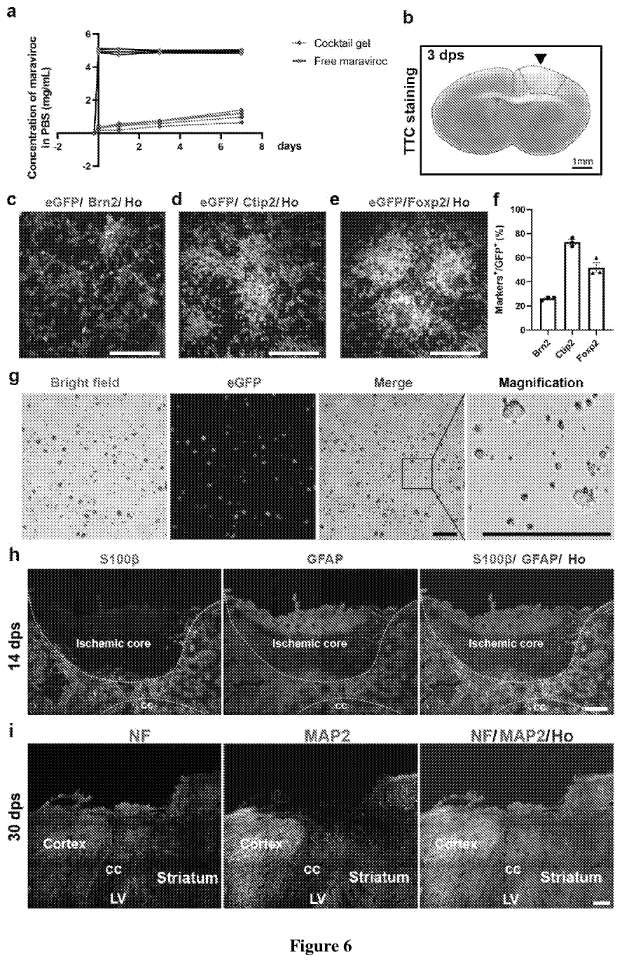

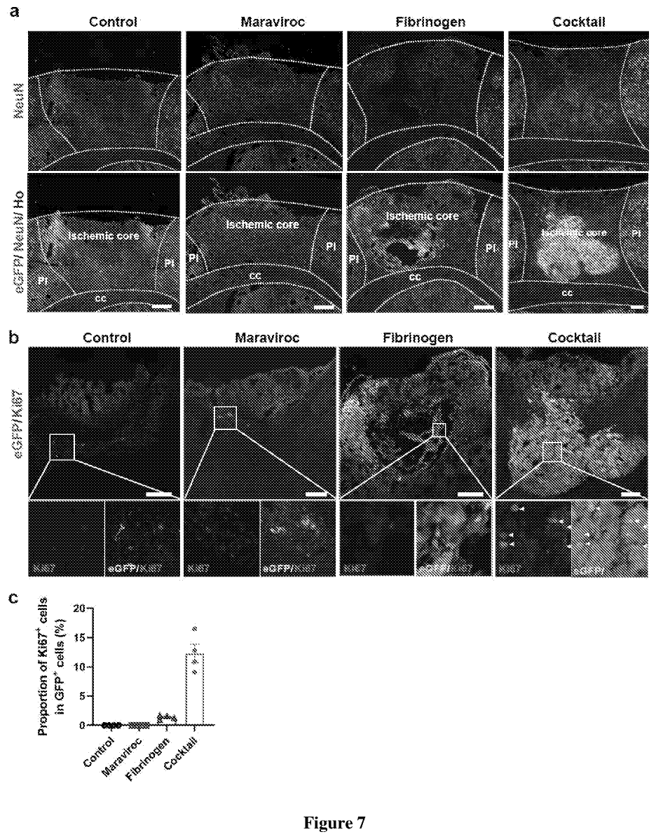

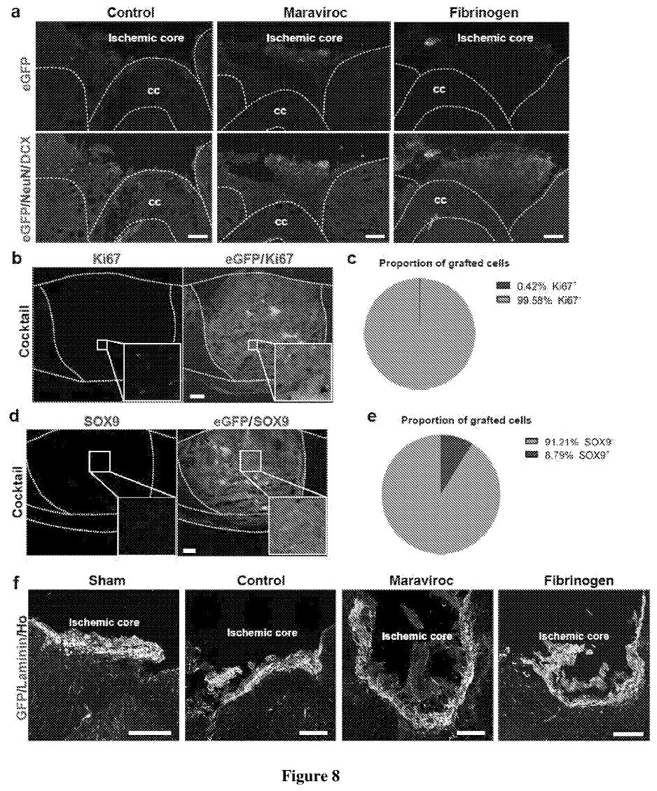

Cell therapy in the present study was targeted for chronic stroke as there is no more spontaneous recovery at this stage. During the chronic phase of stroke, the infarct area forms a lesion cyst or cavity that is walled off by glial scar tissues, and filled with inflammatory cells and their secretions. Such an environment not only lacks physical and nutritional supports for but also exerts inflammatory effect on grafted NPCs. To modify the hostile environment in the infarct core, a cocktail/composition was developed, which consisted of Maraviroc (5 mg/mL) and fibrinogen (9 mg/mL). Maraviroc is an FDA-approved CCR5 inhibitor that specifically blocks the binding between CCR5 and its ligands. To slow the release of Maraviroc, it was mixed with a hydrogel. Fibrinogen (9 mg/mL) was chosen as it retains a soluble state above 0 Degree Celsius, making it easy for transplantation. Upon injection, the fibrinogen mixed with the endogenous thrombin released during surgery and became a gel. Such an injectable gel not only stabilized the grafted cells but also slowed the dilution of Maraviroc, as indicated by the release profile of Maraviroc (FIG. 1a and FIG. 6a). The effect of the cocktail/composition was assessed by transplanting eGFP-H9-derived cortical NPCs (differentiated from human embryonic stem cells (hESCs), the eGFP-H9 hESC line, for 50 days, FIG. 6c-f) directly into the ischemic core at two weeks after stroke in the absence or presence of Maraviroc, fibrinogen, or the cocktail/composition and then the viability of the grafted cells one week later was measured (FIGS. 1b and 1c). The ischemic stroke was induced by photothrombosis in the cerebral cortex of SCID mice in which the ischemic cavity was surrounded by GFAP⁺ and S100β⁺ glial scar at day 14 (FIG. 1c, FIG. 6b-h) and the cortex collapsed at day 30 without treatment (FIG. 6i). The ischemic injury induced by photothrombosis presents a relatively uniform size at a similar location without a penumbral region, offering a consistent model to assessing the efficacy of cell replacement therapy.

Grafted cells, identified by GFP, were observed in the stroke cavity in the presence of fibrinogen only or the cocktail/composition, whereas few or no GFP⁺ cells were observed in the presence of Maraviroc alone or in the cell-only group (FIG. 1c and FIG. 7a). Immunostaining for cleaved-caspase3 revealed strong fluorescence in the control, Maraviroc, and fibrinogen groups with diffuse staining in the control and Maraviroc groups and discrete staining on individual GFP⁺ cells in the ischemic core of the fibrinogen group (FIGS. 1d and 1e), suggesting that the grafted (GFP⁺) cells in the control and Maraviroc groups are dead (fragmented) and the individual GFP⁺ cells in the fibrinogen group are dying. In contrast, few GFP⁺ cells were positive for caspase in the cocktail/composition group (FIGS. 1d and 1e). Thus, the transplanted NPCs survived in the presence of the cocktail/composition.

Stereological quantification of the GFP (overlay on DAPI-labelled nuclei) showed the presence of 1.2×10⁵ cells in the cocktail/composition group (FIGS. 1f and 1g). Over 26% and 81% of grafted cells were SOX2⁺ and DCX⁺, respectively, indicating that most of NPCs were at the immature stage and began to differentiate to neurons (FIGS. 1f and 1g). Immunostaining for Ki67, a marker for cell proliferation, revealed that about 11% of the GFP⁺ cells were Ki67⁺ in the cocktail/composition group, but few in the other three groups (FIGS. 7b and 7c). Together, the results showed that fibrinogen or Maraviroc alone was insufficient to improve the survival of grafted NPCs. In contrast, combination of fibrinogen and Maraviroc supported the survival of the NPCs that were transplanted into the ischemic core.

### Survived NPCs Become Mature Neurons

The milieu in the ischemic core is generally inhibitory to the differentiation of grafted NPCs. To determine if the transplanted NPCs survived for a longer term and what they became, the transplanted brains and the number and fate of the transplanted NPCs were assessed at 30 days post transplantation (FIG. 2a). Grossly, the brains from the sham (stroke without transplantation), control, Maraviroc and fibrinogen groups displayed collapsed cortex with no or few GFP⁺ cells in the ischemic area (FIG. 8a), whereas those from the cocktail/composition group showed a smooth surface that was similar to the contralateral side (FIGS. 2b and 2c). The stroke cavity is walled off by glial scar tissues; hence it can be traced by using GFAP and/or S100β staining. GFP labelled, transplanted cells were observed to be accurately transplanted into the ischemic core, surrounded by GFAP⁺ and S100β⁺ glial scar (FIG. 2b), suggesting that the grafted neurons did not migrate to the peri-infarct area. Strikingly, the transplanted GFP⁺ cells filled the entire stroke cavity at one-month post transplantation (FIG. 2b, 2c). Serial coronal brain sections further revealed that the grafted cells, confirmed by positive staining for the human specific marker STEM121, filled up the infarct area (FIG. 2d), and less than 0.4% of GFP⁺ cells expressed Ki67 (FIGS. 8b and 8c), indicating that transplanted NPCs largely ceased proliferation by one month without overgrowth.

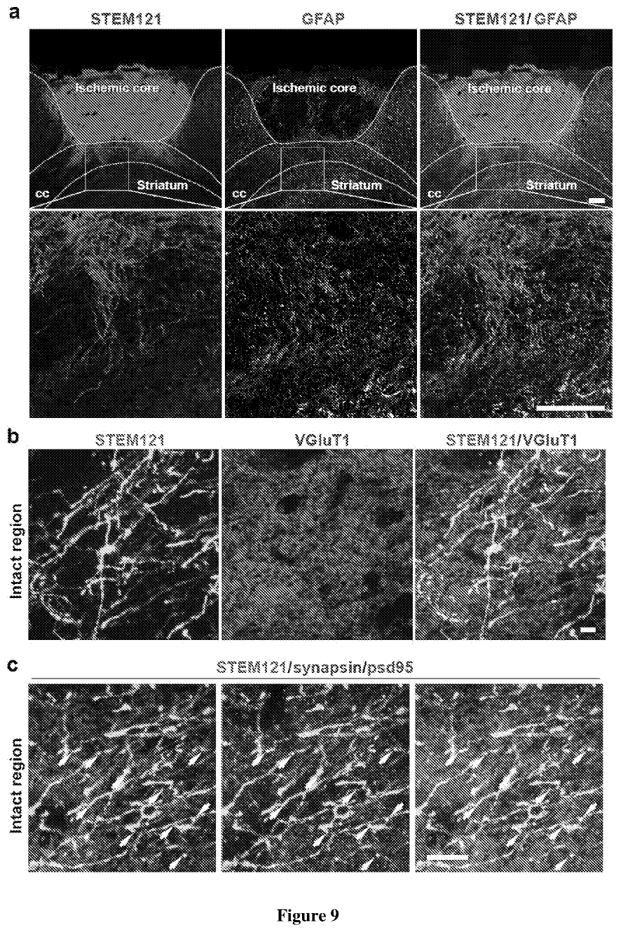

Immunostaining for mature neuronal markers indicated that over 89% of the transplanted cells expressed neurofilament (NF) (FIG. 2e-g). Similarly, 56% of the grafted human (STEM121+) cells were positive for NeuN, another mature neuronal marker (FIG. 2h, 2i). The proportion of NeuN+/STEM121+ cells appeared gradient, with more NeuN+ cells in upper layers and the edge of the stroke cavity than the center in grafts (FIG. 2j-m). About 9% of GFP⁺ cells expressed SOX9, a marker for astroglia or their progenitors (FIGS. 8d and 8e). At this stage, the GFP⁺ cells were largely confined within the boundary of the stroke cavity (FIG. 2e), suggesting that the grafted neurons did not migrate to the peri-infarct area. Similarly, 56% of the grafted human (STEM121⁺) cells were positive for NeuN, another mature neuronal marker (FIG. 2i). The proportion of NeuN⁺/STEM121⁺ cells appeared gradient, with more NeuN⁺ cells in upper layers and the edge of the stroke cavity than the centre in grafts (FIG. 2j-2m). NF is initially expressed corresponding to axon initiation. NeuN is widely used to identify mature neurons with mature axons. Thus, NF is expressed by neurons earlier compared with NeuN. NF and NeuN are specifically expressed by neurons but not glial cells. Although the neurons were localized to the ischemic cavity, their neurites, indicated by positive staining for the human marker STEM121 and glutamatergic neuron marker vGluT1, grew into the undamaged region adjacent to the site of injury (FIGS. 9a and b). Furthermore, the STEM121+ neurites co-expressed a presynaptic marker synapsin and a post-synaptic marker PSD95 (FIG. 9c), demonstrating that the grafted cells develop to mature neurons and form synapses with host neurons. Together, these results indicated that the human NPCs developed to mature neurons in the presence of the cocktail/composition within 30 days.

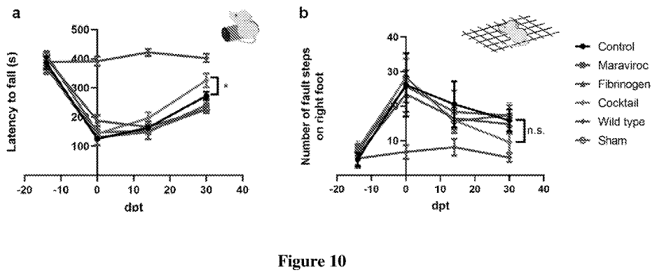

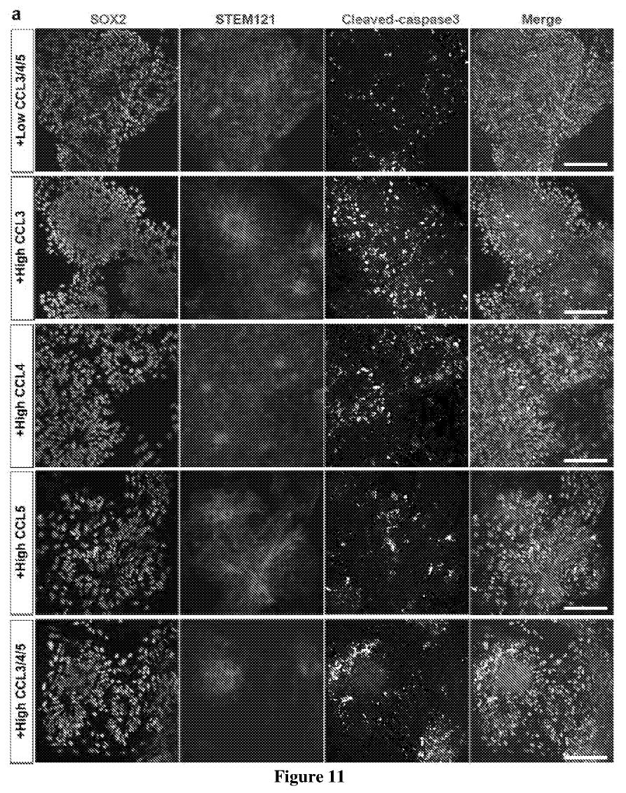

To assess if the cell transplantation contributes to functional improvement, behavioural tests, including the rotarod test and grid-walking test were conducted. While the latency to fall in the rotarod test showed improvement in the cocktail group (FIG. 10a), the grid-walking test did not exhibit significant difference between the cocktail groups and the control groups (FIG. 10b). Hence, a partial functional recovery is achieved at 1 month after NPC transplantation with the cocktail.

Successful Transplantation is Associated with Mitigated Glial Reaction and Restored Vascularization

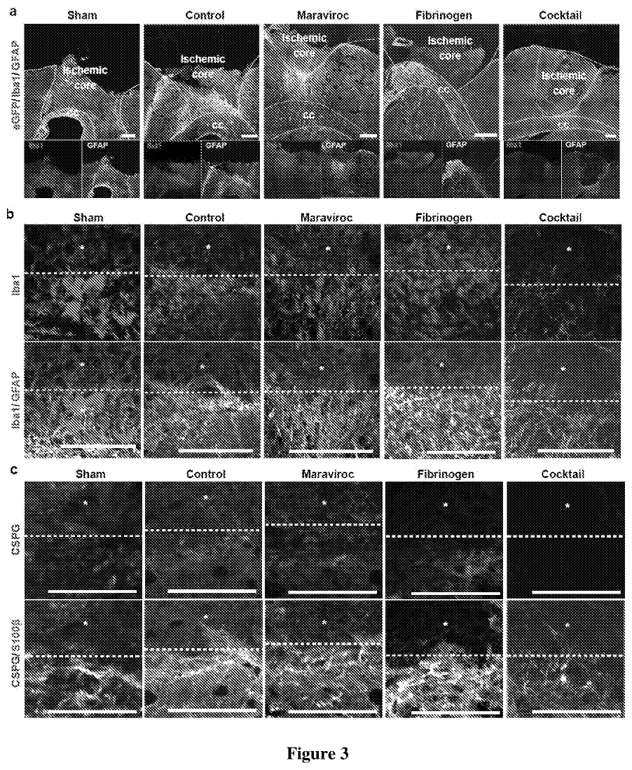

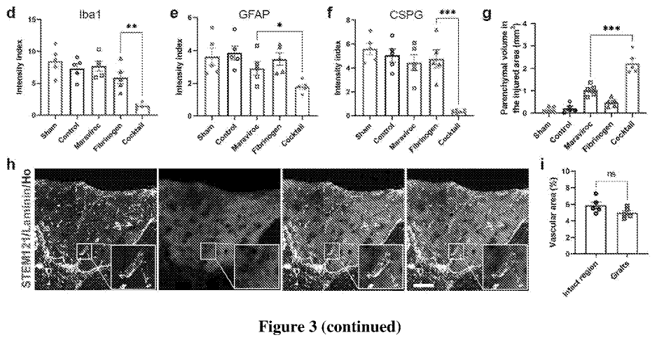

In chronic cerebral stroke, glial scar tissues form around the cavity and the cortex often collapses. In mice that received transplantation without the cocktail/composition, the cortex was collapsed and few or no GFP⁺ cells were present at 30 days after transplantation (FIG. 3a). Strong glial reaction was present in and surrounding the ischemic site, as evidenced by strong staining for a microglial marker Iba1 and an astrocyte marker GFAP (FIGS. 3a and 3b). The Iba1+ microglia/macrophages were present in both the lesion site and penumbra, displaying an ameboid morphology (FIGS. 3b and 3d). The GFAP⁺ reactive astrocytes were accumulated at the peri-infarct to infarct interface (FIGS. 3b and 3e), consistent with the robust deposition of chondroitin sulphate proteoglycan (CSPG) around the ischemic site (FIGS. 3c and 3f). In contrast, in the mice with the cocktail/composition, the ischemic site was filled with GFP⁺ cells so that the ischemic core was not collapsed (FIGS. 3a and 3g). More importantly, substantially fewer activated microglia and reactive astrocytes were accumulated in and around the ischemic site (FIGS. 3d and 3e). The processes of the microglia and astrocytes were thinner (FIG. 3b). The expression of CSPG showed a significantly reduced level at the boundary of the cavity (FIGS. 3c and 3f), indicating the reduction of glial scar.

In the peri-infarct area, vascular remodelling contributes to the neuronal survival after ischemic stroke. Angiogenesis is likely also important for the transplanted cells. By immunostaining for laminin, a membrane protein accumulated in blood vessels, it was found that blood vessels penetrated into the grafts at 30-day post transplantation (FIG. 3h). Compared to the other four groups in which the vasculature was limited to the edge of the lesion (FIG. 8f), the vascular area in grafts showed a similar density to that in the intact cortex (FIG. 3i), suggesting that grafts reconstituted the ischemic cavity with vascularization. These results showed that successful survival of the graft was accompanied by reduced inflammatory response and diminished glial scar as well as restored vascularization.

NPC Transplantation with the Cocktail/Composition Downregulates CCL and CCR5 Expression

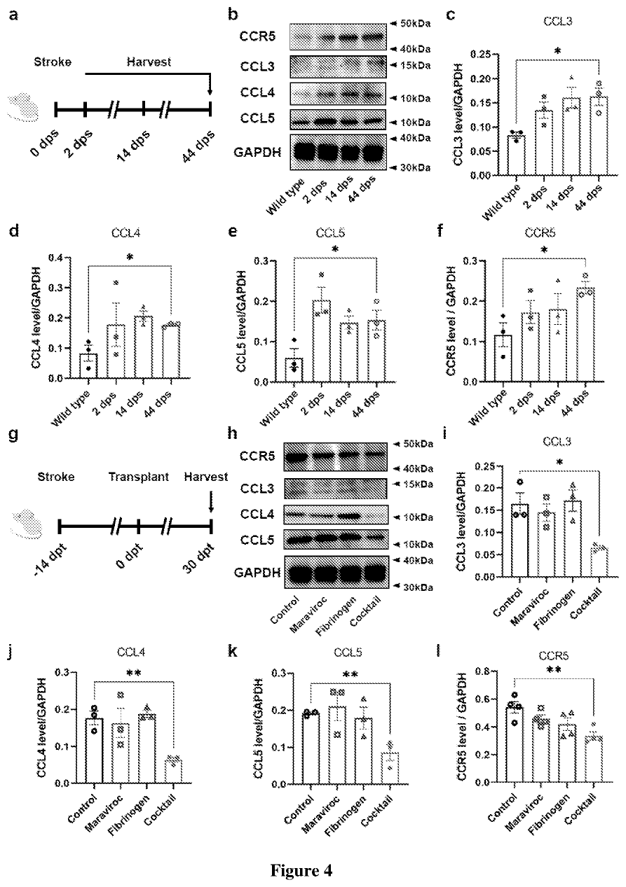

Ischemic injury results in inflammatory response including the production of cytokines and chemokines. Their receptors, including CCR5, are expressed in mature neurons in the peri-infarct area after stroke. CCR5 is one of the receptors for chemokine ligands 3 (CCL3), CCL4 and CCL5. CCL3 and CCL4 are two protein components of macrophage inflammatory protein 1 (MIP), also named MIP1-alpha and beta, respectively. Maraviroc, an antagonist of chemokine receptor CCR5, has been shown to protect the mature neurons bordering the infarct area but not the ischemic core. The expression levels of CCR5 and the three ligands were found to be upregulated in the infarct area after stroke, indicated by Western blotting (FIGS. 4a and 4b). The increased level of CCR5 and its ligands persisted at 44 days after stroke, suggesting that CCR5 was continuously activated with high concentrations of the ligands in the infarct area during the chronic phase (FIG. 4b-4f).

The question then was whether Maraviroc alone, fibrinogen alone, or both Maraviroc and fibrinogen modify the expression of CCR5 or CCL. Western blotting of the transplanted cortical tissues at day 44 after ischemic lesion or 30 days post-transplantation (FIG. 4g) indicated that the CCR5 level was significantly reduced in the cocktail/composition group (FIG. 4h and FIG. 4l). The level of CCL3, 4, 5 did not show obvious difference in the presence of Maraviroc alone or fibrinogen alone but a substantial reduction in the presence of Maraviroc and fibrinogen cocktail/composition (FIG. 4h, 4i, 4j, 4k). The results suggested that Maraviroc alone or fibrinogen alone did not downregulate the CCR5 or CCL3, 4, 5 levels but the transplantation with the cocktail/composition appeared to significantly reduce the presence of CCLs, thus blocking the signalling between CCR5 in injured cortical tissues and CCLs produced by inflammatory cells.

### Blocking the CCR5 Activation Feedback Mitigates Apoptosis of NPCs

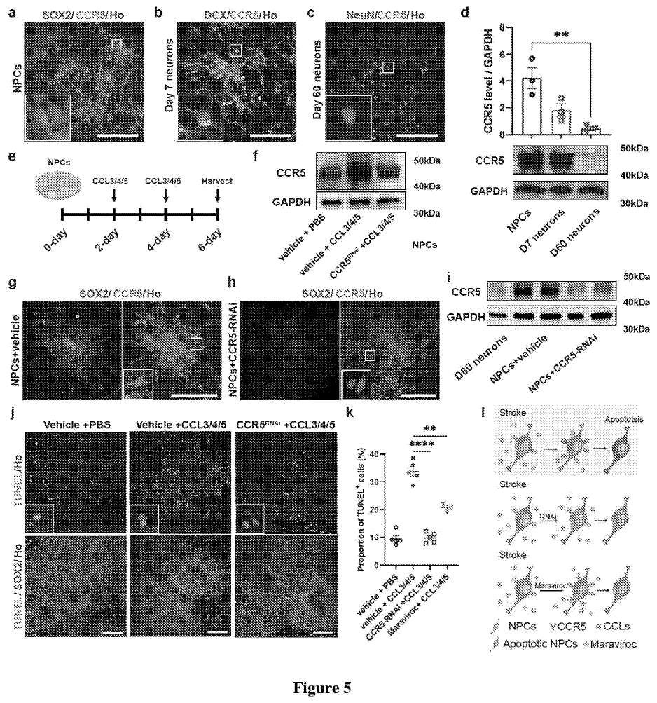

Studies showed that CCR5 was upregulated in neurons in the penumbra area after stroke and blocking the CCR5 signalling by genetic means or Maraviroc (100 mg/kg, i.p. daily) promoted the survival of those neurons and their synaptic connections, thus enhancing the behavioural recovery of animals. This raises a question of how Maraviroc protected the NPCs that were transplanted into the ischemic core. Immunostaining for CCR5 along neural differentiation showed that CCR5 is highly expressed on the membrane and cytoplasm of SOX2⁺ NPCs and DCX⁺ immature neurons (day 7) but the fluorescent signal is significantly diminished in (day 60) mature neurons (FIG. 5a-5c). This was confirmed by Western blotting, showing a progressive reduction of its expression (FIG. 5d). This result suggested that the NPCs and immature neurons were potentially sensitive for the inflammatory chemokines that were present in and surrounding the ischemic cavity.

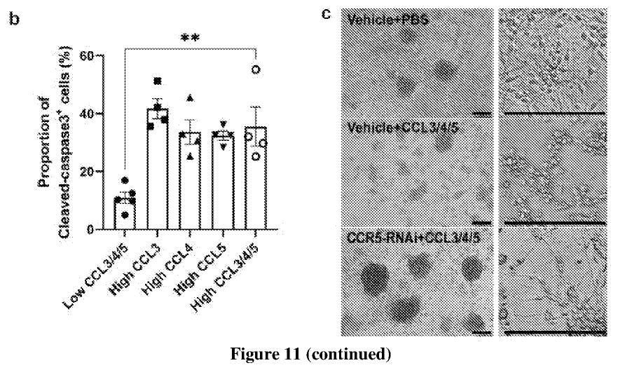

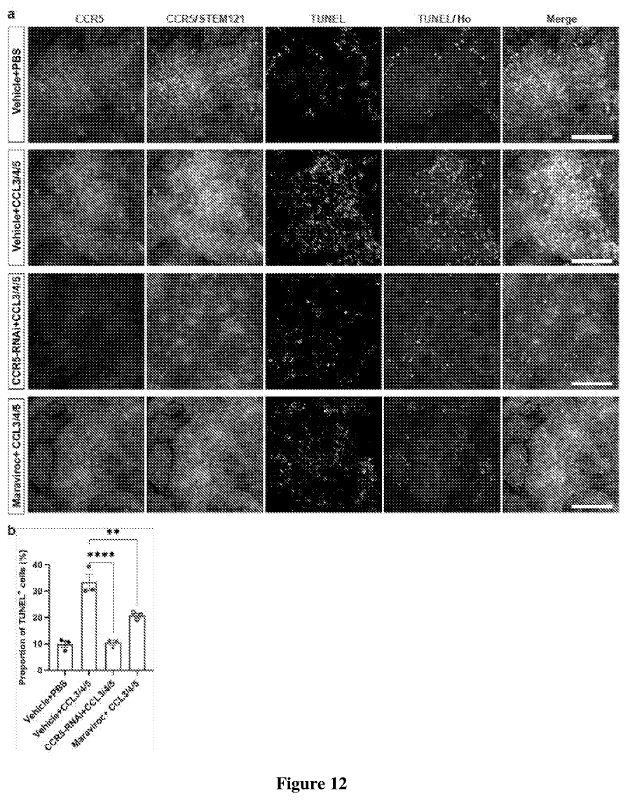

Next, the NPCs were incubated with three ligands (CCL3, CCL4 and CCL5, 300 ng/mL). Compared with the control group, proportion of cleaved-caspase3⁺ cells was increased in all three groups (FIGS. 11a and 11b). To mimic the environment in the ischemic infarction, the combination of three ligands (100 ng/mL each of the three chemokines) was used to incubate the NPCs. Numerous apoptotic and detached neural NPCs were observed after the treatment (FIGS. 11b and 11c). Interestingly, incubation of the differentiating NPCs with the three ligands from day 2 to 4 induced the upregulation of CCR5 in the cells (FIGS. 5e and 5f) and correspondingly increased proportion of TUNEL⁺ cells (FIGS. 5j and 5k). This result suggested that these chemokines indeed promoted the expression of CCR5 on NPCs, inducing apoptosis of NPCs even without microglia.

The next step was to investigate if blockade of the chemokine signalling might mitigate the NPC apoptosis. Lentivirus expressing CCR5-shRNA was used to knock down CCR5 expression. As shown by immunostaining and Western blotting, expression of CCR5-shRNA, but not the control shRNA, significantly reduced the expression of CCR5 in NPCs (FIG. 5g-5i). Correspondingly, the TUNEL⁺ cell population was significantly reduced in cultures that were treated with lentivirus carrying CCR5-shRNA (FIG. 5j-5k and FIG. 12a). Similarly, blocking CCR5 by Maraviroc also reduced the proportion of TUNEL⁺ NPCs without reducing the expression of CCR5 (FIGS. 12a and 12b). Thus, the expression of CCR5 on grafted NPCs might be amplified by the inflammatory milieu in the ischemic cavity and blockade of the CCR5 pathway such as by Maraviroc or RNA interference (RNAi) may protect the susceptible NPCs from apoptosis (FIG. 5l).

Mice with Stroke Transplanted with NPCs Recovered from Motor Deficits

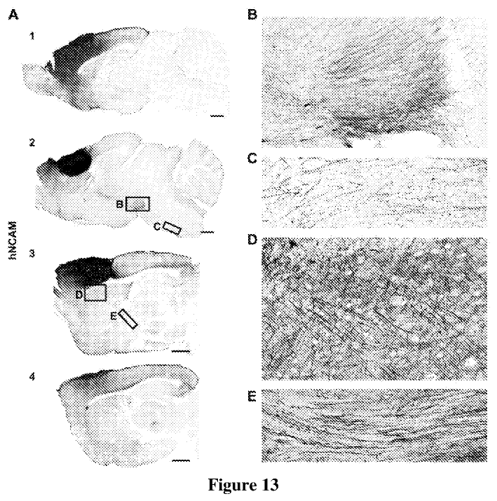

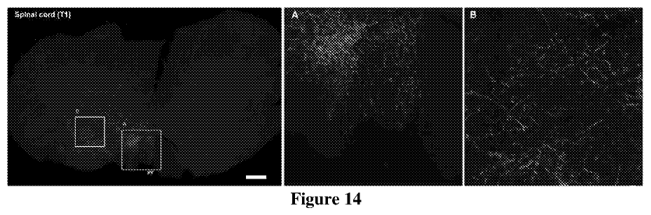

The human ESC-derived neural progenitors were transplanted in the injured site at 14 days after stroke. In rodents, day-14 after stroke is equivalent to the chronic phase in humans. It was shown in the present study that the grafted cells survived and differentiated to neurons at one month after transplantation. With extended time after transplantation, the presence of human axons in the brainstem and spinal cord was observed. Correspondingly, the stroke animals recovered from motor deficits. By 12 months post-transplantation, while the grafted human neurons retained in the stroke region, their axons, labeled by human-specific neural cell adhesion molecule (hNCAM), extended into the striatum (FIG. 13D), traveled through internal capsule (FIG. 13E) and along the pyramidal tract (FIG. 13B). When reaching the medulla, the human axons crossed over to the contralateral side of the pyramidal tract (FIG. 13C). In the spinal cord, the human axons continued to travel down along the contralateral pyramidal tract to as low as at thoracic level. This was evident from the positively immunostained human nerves in the pyramidal tract and neighboring regions (T1) (FIG. 14). These results demonstrated that transplanted human cortical neurons, not only survived and matured at the stroke site, but also grew axons along the cortico-spinal tract all the way down to the spinal cord, thus reconstructing the stroke-damaged cortico-spinal tract. That explained why the stroke animals recovered from the motor deficits.

### Example 3—Discussion

A cocktail/composition consisting primarily of Maraviroc and fibrinogen was developed to support the survival of grafted NPCs in the ischemic core. In the presence of the cocktail/composition, the human NPCs, transplanted into the ischemic cyst, survived, and then divided and matured, reconstituting the injured cortex in the stroke model by day 30. This is achieved by blocking the signalling between inflammatory chemokines in the ischemic lesion and the high level of CCR5 on NPCs. The survival and maturation of the transplanted NPCs in the ischemic core was accompanied by significant attenuation of glial scar and vascularization of the graft. Specifically, the NPCs in the cocktail/composition group survived when assayed at 7 days post-transplantation. Over the following 3 weeks, the surviving NPCs proliferate and fill the stroke cavity. Importantly, most of the transplanted NPCs differentiated into NeuN positive neurons by one month post-transplantation. In the group with fibrinogen alone, there were some surviving cells, which did not proliferate and became GFAP positive astrocytes. These results indicated that the cocktail/composition supported the survival of grafted NPCs in the otherwise hostile environment. Additionally, the host endothelia penetrated into the grafted tissue and formed vasculatures in the graft, supporting the long-term survival and maturation of grafted neurons. Furthermore, glial scar, indicated by elevated expression of GFAP on the boundary of the cavity, was significantly reduced. By 6 months post transplantation, the grafted neurons projected their axons to the brainstem and spinal cord as well as to the contralateral cortex, reconnecting the ischemic cortex with the rest of the brain.

This appeared to be achieved by the cocktail/composition disclosed herein, which supported the survival of NPCs in the inflammatory lesion cavity and promoted the neuronal differentiation of the surviving NPCs. Fibrinogen could form gel and serve as a scaffold to stabilize the grafted cells at the injury site. It also supported the transplanted NPCs growth. Maraviroc, the antagonist of C-C chemokine receptor type 5 (CCR5), blocked signalling between inflammatory chemokines in the ischemic lesion and the high level of CCR5 on NPCs, thus blocked the activation of CCR5 expressed by NPCs, reducing the apoptosis and promoting the maturation of grafted NPCs. The survival and maturation of the transplanted NPCs in the ischemic core was accompanied by significant attenuation of glial scar and vascularization of the graft.

Following ischemic stroke, inflammatory cells infiltrated the lesion and released inflammatory cytokines like CCLs. The reactive glial cells formed a wall around the lesion site to prevent overflow of pro-inflammatory mediators. Hence, the inflammatory milieu in the ischemic cavity persisted, as was observed. Consequently, NPCs transplanted into the cavity rarely survive and the surviving NPCs, if there are, tend to differentiate to astrocytes. It is unclear which inflammatory pathway induces death of the grafted NPCs in the ischemic core. In the penumbra, CCR5 inhibits the expression of PKA and CREB on neurons, leading to an increased loss of dendritic spines and neuronal death. However, this does not explain the low survival of the grafted NPCs in the ischemic core. It was found in the present study that NPCs express a high level of CCR5, highlighting the sensitivity of NPCs to the inflammatory environment. Its ligands, CCL3/4/5, secreted by infiltrating blood-born cells upon stroke, activate microglia and astrocytes. Reactive astrocytes and activated microglia also produce CCL3/4/5 along with other cytokines, forming a cascade of inflammatory response. Together, they induce apoptosis of grafted NPCs that express their receptors CCR5. To make it worse, CCLs, present in the inflammatory environment, further stimulated the expression of CCR5. That explained why NPCs, transplanted into the ischemic cavity, rarely survived. Indeed, no survival of the NPCs that were transplanted into the ischemic core was observed. Therefore, existing conventional experimental cell transplantation therapy mostly targeted the healthy brain region adjacent to the ischemic lesion to avoid the toxic environment in the ischemic lesion. However, transplantation into the penumbra created an additional injury and posed a substantial risk to patients. Thus, there is a need to develop a way to protect the NPCs that are transplanted into the inflammatory ischemic lesion. The cocktail blocks the CCL-CCR5 pathway not only on NPCs directly but also on reactive glia, thus protecting the grafted NPCs directly and indirectly through reduced production of CCLs.

Protection of the grafted NPCs has been primarily aimed at neurotrophic support, such as overexpressing SUMO in NPCs, hypoxic treatment, co-transplantation, and using biomaterials cross-linked with growth factors. However, these methods fail to fill and reconstitute the damaged brain due to limited number of surviving cells. Since inflammation is the leading cause of cell death, blocking the inflammatory signalling may be sufficient to prevent the cells from death. Indeed, it was found that blocking the CCL-CCR5 signalling in NPCs either by a genetic means (RNAi) or a chemical antagonist of CCR5, i.e., Maraviroc, even in the in vitro system, was sufficient to prevent the apoptosis of NPCs. A recent study showed that administration of Maraviroc (100 mg/kg, i.p. daily) rescued the neurons from death in the peri-infarct region. Since there is a lack of blood flow in the ischemic cavity, peripheral administration of medications is unlikely to affect the NPCs grafted in the lesion cyst or cavity. Hence, in this study, the NPCs were transplanted in the presence of Maraviroc but that was not sufficient to rescue any grafted cells. This was possibly due to the rapid dilution and/or degradation of maraviroc (half-life: 14-18 hours). Indeed, when Maraviroc was combined with fibrinogen, which formed a degradable gel in the presence of thrombin or Ca⁺⁺ and which slowed the release of maraviroc, the grafted NPCs survived, even though fibrinogen itself was not shown to support the survival of transplanted NPCs. Fibrinogen is neurotrophic and it is gellable. The fact that fibrinogen alone did not support the survival of NPCs suggested that a simple holding of grafted cells in the lesion cyst or cavity by neurotrophic fibrinogen may be not sufficient for cell survival. Mitigating the inflammatory insult in such an environment may be necessary.

The surviving human NPCs appeared to proliferate and fill the entire lesion cyst or cavity within 30 days post-transplant. Strikingly, the glial scar surrounding the ischemic cavity was greatly reduced, as evidenced by substantial reduction of GFAP and IBA1 immunoreactivity, and vascularization of the graft indicated by laminin-labelled blood vessels occurred. Such a tissue modification was likely the outcome of complex interactions between the transplanted cells and host cells, besides the effect of Maraviroc. Perhaps most strikingly, the vast majority of the transplanted NPCs became post-mitotic neurons within 30 days, indicated by their expression of NeuN and NF. Human cortical NPCs tend to proliferate for a long period before differentiating into mature neurons, which explains the proliferation of surviving NPCs and filling the lesion cyst or cavity within 30 days. The rapid differentiation/maturation may be due to the lack of growth factors in the environment. This is because, it was shown in past studies that in the presence of high concentration of growth factors (thousand folds of their physiological concentrations), spinal NPCs transplanted into the lesion cyst or cavity of injured spinal cord retained the precursor state for several months, leading to overgrowth. Hence, the cocktail/composition disclosed herein offers a base medium for safe cell transplantation therapy, promoting the survival and differentiation of transplanted cells, i.e. NPCs. It may be supplemented with growth factors like FGF2 to promote proliferation, or neurotrophic factors like BNDF and GDNF for further survival and maturation, or notch inhibitors like compound E to enhance cell cycle exit depending on the number and developmental stage of the donor cells and the size of the lesion to repair. While the base cocktail/composition may be modified to fit the need depending on the nature of the disease and the properties of the NPCs, the cocktail/composition disclosed herein opens the possibility to repair the gap lesions like stroke and other inflammatory neurological conditions through cell transplantation therapy.

### INDUSTRIAL APPLICABILITY

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1.-25. (canceled)

26. A composition for supporting survival and differentiation of neural precursor cells (NPCs) grafted into a neurological injury or disease site, the composition comprising:
(a) a gel forming molecule; and
(b) a chemokine receptor type 5 (CCR5) antagonist.

27. The composition of claim 26, wherein the gel forming molecule is selected from the group consisting of:
fibrinogen, agarose, collagen, gelatin, chitosan, alginate, fibrin, hyaluronic acid, laminin, and a degradable polymer selected from the group consisting of:
poly(glycolic acid) (PGA), poly(caprolactone) (PCL), poly(lactic acid) (PLA), poly(lactic acid-co-glycolic acid) (PLGA), and poly(ethylene glycol) (PEG).

28. The composition of claim 27, wherein the gel forming molecule is fibrinogen.

29. The composition of claim 26, wherein the CCR5 antagonist is a small molecule selected from the group consisting of:
Maraviroc, Fuscin, TAK-220, Nifeviroc, DAPTA, Aplaviroc, Aplaviroc hydrochloride, Ophiobolin C, AZD-5672, and Maraviroc-d6, or a nucleic acid selected from the group consisting of:
small interfering RNA (siRNA), small hairpin RNAs (shRNA), and microRNA (miRNA).

30. The composition of claim 29, wherein the CCR5 antagonist is Maraviroc.

31. The composition of claim 26, wherein the NPCs are selected from the group consisting of:
NPCs derived from human embryonic stem cells (ESCs), NPCs derived from induced pluripotent stem cells (iPSCs), embryonic NPCs, and adult NPCs.

32. The composition of claim 26, wherein the neurological injury or disease site is a site resulted from a neurological injury or disease characterized by inflammation, selected from the group consisting of stroke, traumatic brain injury, spinal cord injury, multiple sclerosis, Alzheimer's disease, Parkinson's disease, Huntington disease, and Amyotrophic lateral sclerosis (ALS).

33. The composition of claim 26, wherein the composition further comprises CaCl₂).

34. The composition of claim 26, wherein the composition further comprises thrombin.

35. The composition of claim 34, wherein the thrombin has a concentration of 10 U/mL-500 U/mL.

36. The composition of claim 28, wherein fibrinogen has a concentration of 5 mg/mL-30 mg/mL.

37. The composition of claim 30, wherein the Maraviroc has a concentration of 3 mg/mL-50 mg/mL.

38. A method of treating a neurological injury or disease of a subject, comprising:
(a) mixing NPCs with the composition of claim 26; and
(b) administering a mixture of the NPCs and the composition into a neurological injury or disease site of the subject,
to thereby support survival and differentiation of the NPCs.

39. The method of claim 38, wherein the NPCs are selected from the group consisting of:
NPCs derived from human embryonic stem cells (ESCs), NPCs derived from induced pluripotent stem cells (iPSCs), embryonic NPCs, and adult NPCs.

40. The method of claim 38, wherein the neurological injury or disease is characterized by inflammation and selected from the group consisting of:
stroke, traumatic brain injury, spinal cord injury, multiple sclerosis, Alzheimer's disease, Parkinson's disease, Huntington disease, and Amyotrophic lateral sclerosis (ALS).

41. The method of claim 38, wherein the number of NPCs to be mixed with the composition is 50,000-500,000.

42. The method of claim 41, wherein the number of NPCs to be mixed with the composition is 50,000.

43. The method of claim 38, wherein thrombin is administered to the neurological injury or disease site after the administration of the mixture of NPCs and the composition of claim 26.

44. A kit for use in supporting survival and differentiation of NPCs grafted into a neurological injury or disease site, the kit comprising:
(a) the composition of claim 26;
(b) artificial cerebral spinal fluid (a-CSF);
(c) CaCl₂); and
(d) thrombin.
